SEQUENCE LISTING (38 sequences)

SEQ ID NO: 1 - DNA, 42 bp, Homo Sapien
tcctttgaataatcaaactgaagaaggagaagcaagatgtct

SEQ ID NO: 2 - DNA, 42 bp, Homo Sapien
tcctttgaataatcaaactgaggaaggagaagcaagatgtct

SEQ ID NO: 3 - DNA, 42 bp, Homo Sapien
catcaggattatcagcatttaagccagagttgcaaattaagt

SEQ ID NO: 4 - DNA, 42 bp, Homo Sapien
catcaggattatcagcatttaggccagagttgcaaattaagt

SEQ ID NO: 5 - DNA, 42 bp, Homo Sapien
tgggtccccccgcacagagccatcctgctgccggtagcccgc

SEQ ID NO: 6 - DNA, 42 bp, Homo Sapien
tgggtccccccgcacagagccgtcctgctgccggtagcccgc

SEQ ID NO: 7 - DNA, 42 bp, Homo Sapien
cagaaggaagagttctgggggatcatctggggcctgcagcag

SEQ ID NO: 8 - DNA, 42 bp, Homo Sapien
cagaaggaagagttctgggggctcatctggggcctgcagcag

SEQ ID NO: 9 - DNA, 42 bp, Homo Sapien
atgtgaataataaggataataatcaccaaatacatagacatg

SEQ ID NO: 10 - DNA, 42 bp, Homo Sapien
atgtgaataataaggataatagtcaccaaatacatagacatg

SEQ ID NO: 11 - DNA, 42 bp, Homo Sapien
caagagaatggccactggtcaactaccgtgccaacctgccaa

SEQ ID NO: 12 - DNA, 42 bp, Homo Sapien
caagagaatggccactggtcacctaccgtgccaacctgccaa

SEQ ID NO: 13 - DNA, 42 bp, Homo Sapien
gtcagcacctggaagcccccaatgaaggaaccatggactgtg

SEQ ID NO: 14 - DNA, 42 bp, Homo Sapien
gtcagcacctggaagcccccagtgaaggaaccatggactgtg

SEQ ID NO: 15 - DNA, 42 bp, Homo Sapien
acctgcctagtcctgggcctgacccttgtctttggtgaaggg

SEQ ID NO: 16 - DNA, 42 bp, Homo Sapien
acctgcctagtcctgggcctggcccttgtctttggtgaaggg

SEQ ID NO: 17 - DNA, 42 bp, Homo Sapien
gcctgtaccaatacatcctgcagtggccacggtgaatgtgta

SEQ ID NO: 18 - DNA, 42 bp, Homo Sapien
gcctgtaccaatacatcctgccgtggccacggtgaatgtgta

SEQ ID NO: 19 - DNA, 42 bp, Homo Sapien
acatgtgtacacgtggtgtatattaaaaacttcaggctctct

SEQ ID NO: 20 - DNA, 42 bp, Homo Sapien
acatgtgtacacgtggtgtatgttaaaaacttcaggctctct

SEQ ID NO: 21 - DNA, 42 bp, Homo Sapien
cacaaaggcaacagaccgtgaaaatagatgccaatgtgctag

SEQ ID NO: 22 - DNA, 42 bp, Homo Sapien
cacaaaggcaacagaccgtgagaatagatgccaatgtgctag

SEQ ID NO: 23 - DNA, 42 bp, Homo Sapien
ggtgaagaggctgatggggccaagcaggtcacagagctcatc

SEQ ID NO: 24 - DNA, 42 bp, Homo Sapien
ggtgaagaggctgatggggcccagcaggtcacagagctcatc

SEQ ID NO: 25 - DNA, 42 bp, Homo Sapien
cttcaaaaagtgaaactaactactcgtttctggtaaagagcc

SEQ ID NO: 26 - DNA, 42 bp, Homo Sapien
cttcaaaaagtgaaactaactcctcgtttctggtaaagagcc

SEQ ID NO: 27 - DNA, 42 bp, Homo Sapien
ggcaaagtttcatctattagcaataaaatgtgaattttctgc

SEQ ID NO: 28 - DNA, 42 bp, Homo Sapien
ggcaaagtttcatctattagcgataaaatgtgaattttctgc

SEQ ID NO: 29 - DNA, 42 bp, Homo Sapien
caaaccctttccactccattaaaagaacatgaatcctgataa

SEQ ID NO: 30 - DNA, 42 bp, Homo Sapien
caaaccctttccactccattataagaacatgaatcctgataa

SEQ ID NO: 31 - DNA, 42 bp, Homo Sapien
cccatggccacaacagaactcacaaatggcagagctagggag

SEQ ID NO: 32 - DNA, 42 bp, Homo Sapien
cccatggccacaacagaactcgcaaatggcagagctagggag

SEQ ID NO: 33 - DNA, 42 bp, Homo Sapien
gattttgtcaaagatagattcaggagccatccatttcagagg

SEQ ID NO: 34 - DNA, 42 bp, Homo Sapien
gattttgtcaaagatagattcgggagccatccatttcagagg

SEQ ID NO: 35 - DNA, 42 bp, Homo Sapien
ccctttcagcaacaacaccatcggtagaaatatgatgcagcg

SEQ ID NO: 36 - DNA, 42 bp, Homo Sapien
ccctttcagcaacaacaccatgggtagaaatatgatgcagcg

SEQ ID NO: 37 - DNA, 42 bp, Homo Sapien
gccctggtttcctccagtatgactgcaaaatttcctctccat

SEQ ID NO: 38 - DNA, 42 bp, Homo Sapien
gccctggtttcctccagtatgcctgcaaaatttcctctccat

(19) United States
(12) Patent Application Publication
(10) Pub. No.: US 2007/0202518 A1
(43) Pub. Date: Aug. 30, 2007

(54) Physiogenomic method for predicting statin injury to muscle and muscle side effects

(75) Inventors: Gualberto Ruano, Milford, CT (US); Alan Wu, Palo Alto, CA (US); Paul D. Thompson, Simsbury, CT (US)

Correspondence Address: MORGAN & FINNEGAN, L.L.P., 3 World Financial Center, New York, NY, 10281-2101, US

(73) Assignee: GENOMAS, INC.

(21) Appl. No.: 11/602,108

(22) Filed: Nov. 20, 2006

Related U.S. Application Data
(60) Provisional application No. 60/738,220, filed on Nov. 18, 2005.

(51) Int. Cl. C12Q 1/68; C12M 1/34

(52) U.S. Cl. 435/6; 435/287.2

(57) ABSTRACT

The present invention relates to the use of genetic variants of associated marker genes to predict an individual's susceptibility to muscular injury and muscular side effects in response to statin therapy. The present invention further relates to analytical assays and computational methods using the novel marker gene set. The present invention has utility for personalized medical treatment, drug safety, statin compliance, and prophylaxis of muscle side effect.

This application claims benefit of U.S. provisional application No. 60/738,220, filed Nov. 18, 2005, which is incorporated herein by reference in its entirety.

### FIELD OF THE INVENTION

The present invention is in the field of physiological genomics, hereafter referred to as “physiogenomics”. More specifically, the invention relates to the use of genetic variants of associated marker genes to predict an individual's susceptibility to muscular side effects in response to statin therapy. The present invention further relates to assays and methods using the novel marker gene set. The present invention has utility for personalized medical treatment, drug safety, statin compliance, and muscular side effect prophylaxis.

### BACKGROUND OF THE INVENTION

Hydroxy-methyl-glutaryl (HMG) CoA reductase inhibitors or statins are the most effective medications for managing elevated concentrations of low-density lipoprotein cholesterol (LDL-C). With the understanding of their pleiotropic effects, new indications will arise for statin treatment, including metabolic syndrome (MetSyn), cardiac stabilization, Alzheimer's disease, and osteoporosis. These drugs also offer one of the most effective strategies to reduce cardiovascular disease (CVD) and have been documented to reduce cardiac events in both coronary heart disease patients (Simvastatin Survival Study, 1994; Sacks et al. 1996) and in previously healthy subjects (Shepherd et al. 1995, Downs et al. 1998). Statins are so effective that they are presently the most prescribed drugs in the United States and the world.

Recent guidelines from the National Cholesterol Education Program, the Adult Treatment Panel III, call for more aggressive intervention to prevent cardiovascular disease, not only on serological markers, but also on family history of heart disease and diabetes (NIH 2001). Depending on those risk factors, individualized “target goals” for such a patient is a LDL serum level of 100 mg/dL or less. Indeed, on Jul. 13, 2004, the NCEP stated that an LDL-C goal of <70 mg/dl is “a reasonable clinical strategy” for patients at very high risk of CAD, and that older persons also benefit from LDL-C reduction (Grundy et al. 2004). The expectation is that not only will these guidelines lead to more prevalent use of the statins, but also higher dose, and earlier intervention.

Statins are extremely well tolerated by the majority of patients, but can produce statin injury to muscle (hereinafter “SIM”) and side effects such as, in increasing order of severity, myalgias, cramps, weakness, tenderness, wasting, myositis, myopathies, and rhabdomyolysis. The reported incidence of myalgia during therapy with the more powerful statins has varied from 1% in pharmaceutical company reports (PDR 2002) to 25% (Phillips et al. 2002) of patients. In clinical practice, approximately 5% of the patients develop myalgias severe enough to trigger a referral to a specialist or provoke a change in drug treatments. In addition, weakness is a clinically acknowledged complication of statin use, but generally not addressed or well defined in the medical literature. The widespread use of statins may lead to unsafe serum levels in patients treated at high dose, compromised by frailty or co-medicated with other drugs inhibiting statin excretion. As treatment goals become more aggressive, and statins are utilized in increasingly younger patients with disease risk factors, the side effect risk factors may begin to balance the choice of treatment.

The major risk of these drugs is myositis with rhabdomyolysis and possible acute renal failure and even death. Skeletal muscle weakness is frequently associated with clinically important myositis and rhabdomyolysis, but can also occur in patients with no or little creatine kinase (CK) elevation. Although labeled as “mild muscle complaints”, myalgia, cramps, and weakness are critically important side effect because they limit use of these drugs and because weakness affects mobility and injury risk in older subjects. Rhabdomyolysis, a potentially lethal manifestation of statin toxicity, has recently served to alert the medical community about the safety aspects of statin therapy, as exemplified by the withdrawal of cerivastatin in August, 2001, after the drug was associated with approx. 100 rhabdomyolysis-related deaths. Fortunately, clinically important rhabdomyolysis with statins is exceedingly rare with an overall reported incidence of fatal rhabdomyolysis of 0.15 deaths per 1 million prescriptions (Staffa et al. 2002). Nevertheless, the safety concerns delayed regulatory approval of rosuvastatin (Crestor®, AstraZeneca). Thus, there is considerable medical value to the DNA diagnostics in this patent to diagnose whether the subjective symptoms are likely to have at least mechanistically plausible causes.

Physiogenomics integrates genotype, phenotype, and population analysis of functional variability among individuals. In physiogenomics, genetic markers (e.g. single nucleotide polymorphisms or “SNPs”) are analyzed to discover statistical associations to physiological characteristics or outcomes in populations of individuals either at baseline or after they have been exposed to an environmental trigger such as a drug. Variability in a genomic markers among individuals that corresponds to variability in physiological characteristics establishes associations and mechanistic links with specific genes.

There is a need for better understanding of the mechanisms of SIM. Genetic understanding in particular can be translated into DNA diagnostics for safe prescription of the drugs based on identification of patients at high risk for the commonly observed statin complications. These diagnostics would be most useful products to guide medical management of statins by dose reduction, alternative drug selection, avoidance of interacting drugs, or dietary supplementation with ubiquinone (Coenzyme Q10, CoQ10).

### SUMMARY OF THE INVENTION

The present invention, in its broadest aspect, relates to a physiogenomic method for determining an individual's risk of muscle injury and/or muscular side effects in response to statin therapy. The method utilizes physiogenomics to identify gene variants of associated marker genes, i.e. an array of marker genes, whose presence has been newly found to be associated with statin-induced muscular injury and/or muscular side effects.

In one aspect, the present invention provides a method of determining an individual at risk for muscle injury and/or muscular side effects in response to statin treatment by determination of a genetic variant of a marker gene associated with the increase risk of SIM during statin therapy, where the presence of the genetic variant is indicative of a risk factor for muscle injury and/or muscular side effects during statin therapy.

In a specific aspect, the marker genes newly associated with statin response include, but are not limited to, angiotensin II Type 1 receptor (AGTR1) and nitric oxide synthase 3 (NOS3). Identification of genetic variants of these marker genes, is indicative of a risk factor for muscle injury and/or muscular side effects during statin therapy.

Another specific aspect of the method involves obtaining nucleic acid, e.g. DNA, from a subject, and assaying the DNA to determine if there is a specific gene variant of one or a combination of the marker genes that have been newly discovered to be associated with muscle injury caused by statin treatment. Micro- and nano-array analysis of the DNA is preferred in this specific aspect of the invention.

In another aspect, the present invention further provides a method for the development of novel diagnostic systems, termed “physiotypes”, which are developed from combinations of gene polymorphisms and baseline characteristics, to provide physicians with individualized patient risk profiles for statin-induced muscle injury and/or muscular side effects for the management of dyslipidemias.

Yet another aspect of the present invention provides a system containing a support or support material, e.g. a micro- or nano-array, comprising a novel set of marker genes and/or gene variants newly associated with statin-induced muscular side effects in a form suitable for the practitioner to employ in a screening assay for determining an individual's genotype. In addition to the marker genes and gene variants, the system comprises an algorithm for predicting the risk based on a predetermined set of mathematical equations providing specific coefficients to each of the components of the array.

In another aspect, the present invention provides methods for the identification of a population of individuals that are susceptible to muscle injury in response to statin therapy. These individuals, who are identified through screening using the methods of the present invention, are especially amenable to specific treatments or therapies to reduce the occurrence of muscle injury and/or muscular side effects in response to statin therapy.

A further aspect of the present invention is to provide a means to identify a population of patients at risk for muscular side effects caused by statin treatment to be used as a population to test and evaluate substances, e.g. compounds or drugs, to identify those substances that prevent or reduce the muscular side effects caused by statin treatment. In accordance with the present invention, such substances that prevent or reduce muscle injury caused by statin treatment are suitable for use in muscle side effect prophylaxis. Thus, the present invention also provides a method for screening for a desired prophylactic or therapeutic compound by determining if the compound prevents or reduces muscular side effects caused by statin treatments in one or more individuals identified to be at risk for such side effects.

In one embodiment, coenzyme Q10 (CoQ10) supplementation is a useful preventive measure for satin induced muscle injury. CoQ10 is a coenzyme for the inner mitochondrial enzyme complexes involved in oxidative phosphorylation. Satins, particularly at higher doses, interfere with CoQ10 synthesis by blocking HMG-CoA-reductase and thus result in reduced serum CoQ10 levels. While not being bound by a particular mechanism of action, muscle injury may be triggered by low Q10 levels.

In a related embodiment, the present invention provides a means to identify a population of patients at risk for muscular side effects caused by statin treatment to be used as a population for analyzing the mechanism of statin action on muscle to determine potential targets to interfere with those actions. The method employs one or more of the newly identified gene variants that have been discovered to be associated with muscular side effects during statin treatment as described herein.

These and other aspects of the present invention will be better understood upon a reading of the following detailed description when considered in connection with the accompanying figures.

### BRIEF DESCRIPTION OF THE DRAWINGS

FIG. 1. Distribution of log(CK), with the reference range indicated.

FIG. 2. Individual genotypes (circles) of each SNP overlaid on the distribution of log (CK) (thin line). Each circle represents a subject, with the horizontal axis specifying the log(CK), and the vertical axis the genotype: bottom—homozygous for major allele, middle—heterozygous, top—homozygous for minor allele. A LOESS fit of the allele frequency as a function of log(CK) (thick line) is shown.

FIG. 3. Receiver Operator Characteristic (ROC) curve by the number of independent physiogenomic factors with modest associations with the outcome (OR=1.7), and the target area (grey).

FIG. 4. Power calculations for detecting an genotype effect in 288 subjects by gene prevalence and size of the effect (OR).

FIG. 5. Power available for a study with 210 subjects with available data on creatine kinase which has a log normal distribution with standard deviation of the natural log equal to 0.44 using 5% level of significance, by percent change and gene prevalence.

### DETAILED DESCRIPTION OF THE INVENTION

The present invention relates to a physiogenomic method for determining an individual's risk of muscle injury and/or muscular side effects in response to statin therapy. The methods described herein utilize physiogenomics to identify gene variants of associated marker genes whose presence have been newly found to be associated with statin-induced muscular injury and/or muscular side effects.

Molecular markers to assess the level of muscle injury can be used to identify the gene variants of associated marker genes whose presence is associated with statin-induced muscular injury and/or muscular side effects. In one embodiment, serum creatine kinase (CK) levels are used to assess the degree of muscle injury (Staffa et al 2002). In another embodiment, the BB-type CK isoenzyme activity is used to assess the degree of muscle injury. Normally absent in serum, the BB-type CK isoenzyme is preferentially distributed in smooth muscle (Kato et al 1985). Despite the energetic flux being much lower in smooth muscle compared to striated muscles, CK-BB has been found present and active in all smooth muscles studied to date. The CK-BB system responds to pathological insults and development by changes in sub-cellular distribution localization and specific activity (Clark 1994). Smooth muscle neoplasms have given raise to CK-BB in serum (Hoag et al 1980).

In one embodiment, the present invention provides a method of determining an individual at risk for muscle injury and/or muscular side effects in response to statin therapy by determination of a genotype newly identified as associated with the statin-induced muscle injury, where the presence of the genotype is indicative of a risk factor for muscle injury and/or muscular side effects in response to statin therapy.

In some embodiments, genotypic variants in genes associated with endothelial homeostasis are used to determine if a patients is at risk for developing statin-induced muscular injury and/or muscular side effects. Genes associated with endothelial homeostasis include, but are not limited to, AGTR1 (angiotensin II receptor, type 1, encodes the type 1 receptor for angiotensin II, a key vasopressor hormone, and mediates the major cardiovascular effects of angiotensin II (Murphy et al 1991)); NOS3 (nitric oxide synthase 3, produces nitric oxide from L-arginine in endothelial cells. Nitric oxide (NO) is an inhibitor of smooth muscle contraction and platelet aggregation (Zoellner et al 1997)); ANGPT1 (angiopoietin1, plays a central role in mediating synergistic interactions between the endothelium and the surrounding matrix and mesenchyme (Carmeliet et al 2003)); OXT (oxytocin, a peptide hormone involved in contraction of smooth muscle during parturition and lactation); FLT1 (FMT-related tyrosine kinase 1, a tyrosine kinase receptor for vascular endothelial growth factors (Kendall et al 1996)); EDN1 (endothelin 1, plays a role in regulation of vascular tone and smooth muscle contraction (Ahn et al 2004)); SELP (selectin P, mediates leukocyte interaction with platelets and endothelial binding after vascular injury); SELE (selectin E, expressed by cytokine-stimulated endothelial cells and mediates the adhesion of cells to the vascular lining); OLR1 (oxidized low-density lipoprotein receptor, encodes a receptor protein which binds, internalizes and degrades oxidized low-density lipoprotein (Imanishi et al 2002, D'lntrono et al 2005)); SERPINE1 (serpin peptidase inhibitor clade-E, inhibits the activity of plasminogen activator A and may play a role in endothelial cell adhesion (Marshall et al 2003)).

In more specific embodiments, genotypic variants in the angiotensin II Type 1 receptor (AGTR1), nitric oxide synthase 3 (NOS3), fms-related tyrosine kinase 1 (FLT1), and apolipoprotein A-IV (APOA4) are used to determine if a patients is at risk for developing statin-induced muscular injury and/or muscular side effects. The specific variants comprising the newly identified marker gene set are presented in Tables 3, 4, 6 and 7. According to the present invention, one, all, or a combination of these genes can be employed as a unique marker, or risk factor, for statin-induced muscle injury and/or muscular side effects.

As stated above, statins are prescribed in increasing numbers and at higher doses to individuals as a treatment for dyslipidemia. Therefore, the methods and assays described herein provide physicians with individualized risk profiles for statin-induced muscular side effects for the management of dyslipidemias and to improve statin safety. SIM, statin muscle injury, as the term is used herein, includes, but is not limited to, muscle weakness, myalgia, cramps, myositis, myopathy, rhabdomyolysis, muscle toxicity, as well as increases or decreases in the expression of proteins or enzymes directly or indirectly involved in muscle metabolism or homeostatis such as, for example, without limitation, creatine kinase (CK). In addition, the identification of genes and metabolic pathways contributing to the statin-induced muscle injury, reaction in individuals will permit the identification of compounds that can prevent or inhibit these muscular side effects, as well as facilitate the development of lipid lowering drugs that do not produce myopathies.

In a related embodiment, the resulting novel genotypes can further be developed into “physiotypes” from combinations of contributory gene polymorphisms and baseline physiologic characteristics. Physiotypes are predictive models incorporating genotypes from various genes and any covariates (e.g. baseline serum levels and clinical examination) and integrate the combined information of genotype and phenotype. Physiotypes are derived from different genes in interacting pathways, which allow sampling of the genetic variability in entire physiological networks. Although an individual's genotype does not change, other physiological characteristics may influence the individual's phenotype and may alter the physical response to statin therapy based on interacting physiological pathways. Physiotypes have utility in personalized medical treatment and facilitate the assessment of accurate risk-benefit-ratios for medical management of dyslipidemias.

One embodiment of the present invention involves obtaining nucleic acid, e.g. DNA, from a blood sample of a subject, and assaying the DNA to determine the individuals' genotype of one or a combination of the marker genes associated statin response. Other sampling procedures include but are not limited to buccal swabs, saliva, or hair root. In a preferred embodiment, genotyping is performed using a gene array methodology, which can be readily and reliably employed in the screening and evaluation methods according to this invention. A number of gene arrays are commercially available for use by the practitioner, for example, but not limited to, static (e.g. photolithographically set), suspended (e.g. soluble arrays), and self assembling (e.g. matrix ordered and deconvoluted). More specifically, the nucleic acid array analysis allows the establishment of a pattern of gene expression variability from multiple genes and facilitates an understanding of the complex interactions that are elicited in an individual in response to a drug or treatment, such as statin therapy.

In a specific embodiment, the array consists of several hundred genes and is capable of genotyping hundreds of DNA polymorphisms simultaneously. Candidate genes for use in the arrays of the present invention are identified by various means including, but not limited to, pre-existing clinical databases and DNA repositories of myalgia cases, review of the literature, and consultation with clinicians, differential gene expression models of myopathy, physiological pathways in statin metabolism, cholesterol and lipid homeostasis, mitochondrial energy production, apoptosis, inflammation, and muscle contraction and repair, and from previously discovered genetic associations. In a preferred embodiment, the candidate genes are selected from those shown in Table 7. The gene array includes all of the novel marker genes, or a subset of the genes, or unique nucleic acid portions of these genes. The gene array of the invention is useful in discovering new genetic markers of susceptibility to statin-induced muscular side effects including, but not limited to, myalgias, myopathy and elevated CK.

In another embodiment, the present invention provides a screening method to allow the identification of subsets of individuals who have specific genotypes and physiological characteristics and are susceptible to muscle injury in response to statin therapy. For example, a screening method of this embodiment involves obtaining a sample from an individual undergoing testing, such as a blood sample, e.g. as described in example 1, and employing an assay method, e.g. the array system and newly-identified marker genes and gene variants as described, to evaluate whether the individual has a genotype associated with statin-induced muscle injury. In a specific embodiment, more than one SNP is used to determine if a patient is at risk for developing muscle injury in response to statin therapy. In a more specific embodiment, the more than one SNP comprises at least one SNP with a positive coefficient and at least one SNP with a negative coefficient. The physigenomics method of the invention mathematically assigns to each SNP a coefficient according to pre-established rules and covariates. The generation of the coefficients is discussed in detail in the examples and in U.S. patent application Ser. No. 11/371,511 and U.S. patent application Ser. No. 11/010,716, both of which are incorporated by reference herein. The coefficient for each SNP may be either positive, indicating that the presence of that marker contributes to physiological response, or negative (i.e., a torpid marker). The most powerful predictions are achieved for a particular physiological endpoint by using SNPs having positive coefficients and SNPS having negative coefficients.

Individuals identified through screening using the methods of the present invention would be especially amenable to specific treatments, therapies, or further study to reduce the occurrence of muscle injury in response to statin therapy.

Yet another embodiment of the present invention is to identify a population of patients at risk for muscular side effects caused by statin treatment to be used as a population to test and evaluate substances, e.g. compounds or drugs, to identify those substances that prevent or reduce the muscular side effects caused by statin therapy. In accordance with the present invention, such substances that prevent or reduce muscle injury caused by statin treatment are suitable for use in muscle side effect prophylaxis. The evaluation method employs one or more of the newly identified gene variants that have been discovered to be associated with muscular side effects during statin treatment as described herein. Thus, the present invention also provides a method for screening for a desired prophylactic or therapeutic compound by determining if the compound prevents or reduces muscular side effects caused by statin treatments in one or more individuals identified to be at risk for such side effects.

In another embodiment, a diagnostic kit containing a support or support material, such as, without limitation, a nylon or nitrocellulose membrane, bead, or plastic film, or glass, or micro- or nano-array, comprising the novel set of genes as described herein, in a form suitable for the practitioner to employ in screening individuals. The kit can contain the novel gene marker set associated with an increased risk for statin-induced muscle injury, or a subset of these genes, on a suitable substrate or micro- or nano-array. In addition, the kit can optionally contain other materials necessary for carrying out the assay method, including, but not limited to, labeled or unlabeled nucleic acid probes, detection label, buffers, controls, and instructions for use.

In a specific embodiment, an ensemble of marker genes useful for determining an individual at risk for muscle injury and/or muscular side effects in response to statin treatment comprising at least two single nucleotide polymorph (SNP) gene variants selected from the group consisting of: rs2933249; rs12695902; rs1549758; rs1799983; rs1800808; rs6136; rs6131; rs6092; rs5361; rs2742115; rs5369; rs877172; rs1283718; rs2514869; rs1283694; rs1570679; rs2296189; rs10507383; rs748253; rs675; rs2740574; rs1800716; rs2020933; rs2070424; rs854572; rs3756450; rs600728; rs3176921; rs10841044; rs7200210; rs5491; rs617333; rs2058112; rs1800794; rs504714; rs6195; rs1042718; rs2276307; rs7412; rs6488950; rs9904270; rs2049045; rs6265; rs132653; rs6318; and rs2838549.

In another specific embodiment, an ensemble of marker genes useful for determining an individual at risk for muscle injury and/or muscular side effects in response to statin treatment comprising:

at least two SNP gene variants, the presence of which correlates with at least one statin injury to muscle and muscle side effects in humans;

wherein said injury is selected from the group consisting of log concentration of serum creatine kinase and myalgia; and combinations thereof; and

(a) in the case where said injury is the log concentration of serum creatine kinase, said ensemble of marker genes comprises rs1799983; rs877172; rs675; rs12695902; rs2740574; rs1800716; rs2020933; rs2296189; rs2070424; rs854572; rs3756450; rs1611115; rs600728; rs3176921; rs10841044; rs7200210; rs5491; rs617333; rs1549758; and rs2514869; and

(b) in the case where said injury is myalgia, said ensemble of marker genes comprises rs2058112; rs1800794; rs504714; rs6195; rs2742115; rs1042718; rs2276307; rs7412; rs6488950; rs9904270; rs1570679; rs2049045; rs6265; rs132653; rs6318; and rs2838549.

The following example demonstrates preferred embodiments of the invention. It should be appreciated by those of skill in the art that the techniques disclosed in the example which follows represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The content of all patents, patent applications, published articles, abstracts, books, reference manuals, sequence accession numbers, as cited herein are hereby incorporated by reference in their entireties to more fully describe the state of the art to which the invention pertains.

### EXAMPLE 1

### Physiogenomic Analysis Links Serum CK Activities During Statin Therapy to Vascular Smooth Muscle Homeostasis

Statins are extremely well tolerated by the majority of patients, but can produce a variety of muscular complaints ranging from mild myalgia to frank rhabdomyolysis. (Thompson et al 2003). Serum creatine kinase (CK) levels are used clinically to assess the degree of muscle injury, although myalgia and skeletal muscle weakness can occur in patients with no or little CK elevation. In the clinically rare condition of rhabdomyolysis the relationship between statin-induced muscle injury, extremely elevated CK and clinical severity is well established (Staffa et al 2002).

Various mechanistic hypotheses have been posited to explain statin-induced muscle injury (Rosenson 2004). These hypothesis range from pharmacodynamics, e.g. interference with energy transduction process by statin interactions with HMG-CoA reductase homologue proteins, to pharmacokinetics, e.g. variability in drug metabolism by the cytochrome p450 system (Wilke 2005). One of the most intriguing hypotheses, described by Thompson et al (2003), involves apoptosis and extends beyond the realm of classical pharmacodynamics and pharmacokinetics. The hypothesis, based on cell culture studies, is that statins induce apoptosis in vascular smooth muscle (Guijarro et al 1998, Knapp et al 2000).

Physiogenomics were used as a technique to explore the vascular hypothesis. Physiogenomics is a medical application of sensitivity analysis (Ruaño et al 2005, Saltelli et al 2000). Sensitivity analysis is the study about the relations between the input and the output of a model and the analysis utilizing systems theory, of how variation of the input leads to changes in output quantities. Physiogenomics utilizes as input the variability in genes, measured by single nucleotide polymorphisms (SNP) and determines how the SNP frequency among individuals relates to the variability in physiological characteristics, the output. The results suggest that statins may affect vascular smooth muscle function.

Genetic associations with serum creatine kinase (CK) levels were screened in 102 patients receiving statin therapy for hypercholesteremia to find physiologic factors influencing statin muscle toxicity. A total of 19 single nucleotide polymorphism (SNPs) were selected from 10 candidate genes involved in vascular homeostasis. Multiple linear regression was used to rank the SNPs according to probability of association, and the most significant associations were analyzed in greater detail. SNPs in the angiotensin II Type 1 receptor (AGTR1) and nitric oxide synthase 3 (NOS3) genes were significantly associated with CK activity. These results demonstrate a strong association between CK activity during statin treatment and variability in genes related to vascular function, and suggested that vascular smooth muscle function contributed to the muscle side effects of statins.

Materials and Methods

Patient enrollment. Patients treated with statins for at least 1 month were recruited from Hartford Hospital clinics and provided informed written consent as approved by the Hartford Hospital Institutional Review Board. All patients were recruited and entered into the study by one investigator (AW). Subjects were not included if they were on a statin other than atorvastatin or simvastatin or were on multiple lipid lowering medications. Valid genotype data were obtained on 102 patients (Table 1). Statin name and dose were obtained by self-report. Clinical laboratory data including lipid profiles, CK values and liver function tests were obtained from the most recent medical records.

Laboratory analysis. Blood was either prospectively collected or retrieved from attending physician-ordered routine clinical analysis. Samples were collected into tubes containing either EDTA or citrate for DNA extraction. The blood was centrifuged, and the plasma was assayed within 2 days for total CK activity using the Cobas Integra Analyzer (Roche Diagnostics, Indianapolis, Ind.). The reference range was <200 U/L for males and <140 U/L for females. The DNA was extracted from leukocytes in 1 ml of whole blood using the Puregene Gentra DNA isolation kit.

Gene selection and description. Ten candidate genes were broadly selected for their role in various processes in endothelial homeostasis including vascular contraction and dilation, apoptosis, cell adhesion, and maturation. The candidate genes were AGTR1, NOS3, ANGPT1, OXT, FLT1, EDN1, SELP, SELE, OLR1, and SERPINE1.

Genotyping Technology and Assay. Genotyping was performed using the Illumina BeadArray™ platform and the GoldenGate™ assay (Oliphant et al 2002, Fan et al 2003). Table 2 lists the assay information and observed allele frequencies for the SNPs used in this study.

Data Analysis. CK activities were log transformed to obtain an approximately normally distributed variable log(CK). Covariates were analyzed using multiple linear regression and the stepwise procedure. Of the potential covariates Age, Gender, Race, Statin, and Dose, only Age was significantly associated with log (CK). An extended linear model was constructed including the significant covariate (Age) and the SNP genotype. SNP genotype was coded quantitatively as a numerical variable indicating the number of minor alleles: 0 for major homozygotes, 1 for heterozygotes, and 2 for minor homozygotes. The F-statistic p-value for the SNP variable was used to evaluate the significance of association. Table 3 lists all SNPs that were tested and their association p-values. The validity of the p-values were tested by performance of an independent calculation of the p-values using permutation testing. The ranking of the first four SNPs were identical under permutation and F-statistic analyses (data not shown). To account for the multiple testing of 19 SNPs, adjusted p-values were calculated using Benjamini and Hochbergs false discovery rate (FDR) procedure (Reinere et al 2003, Benjamini et al 1995, Benjamini and Hochberg 2000). In addition, the power for detecting an association based on the Bonferroni multiple comparison adjustment was evaluated. For each SNP, the effect size in standard deviations that was necessary for detection of an association at a power of 80% (20% false negative rate) was calculated using the formula

$$Δ = \frac{{z}_{a / c} + {z}_{β}}{\sqrt{Nf ( 1 - f )}} ,$$

where α was the desired false positive rate (α=0.05), β the false negative rate (β=1−Power=0.2), c the number of SNPs, z a standard normal deviate, N the number of subjects, f the carrier proportion, and Δ the difference in log(CK) between carriers and non-carriers expressed relative to the standard deviation (Rosner 1995).

LOESS representation. A locally smoothed function of the SNP frequency as it varies with log(CK) was used to visually represent the nature of an association. LOESS (LOcally wEighted Scatter plot Smooth) is a method to smooth data using a locally weighted linear regression (Cleveland 1979; Cleveland and Devlin 1988). At each point in the LOESS curve, a quadratic polynomial was fitted to the data in the vicinity of that point. The data were weighted such that they contributed less if they were further away, according to the following tricubic function where x was the abscissa of the point to be estimated, the xᵢ were the data points in the vicinity, and d(x) was the maximum distance of x to the xᵢ.

$${w}_{i} = {( 1 - {\vert \frac{x - {x}_{i}}{d ( x )} \vert}^{3} )}^{3}$$

### Results

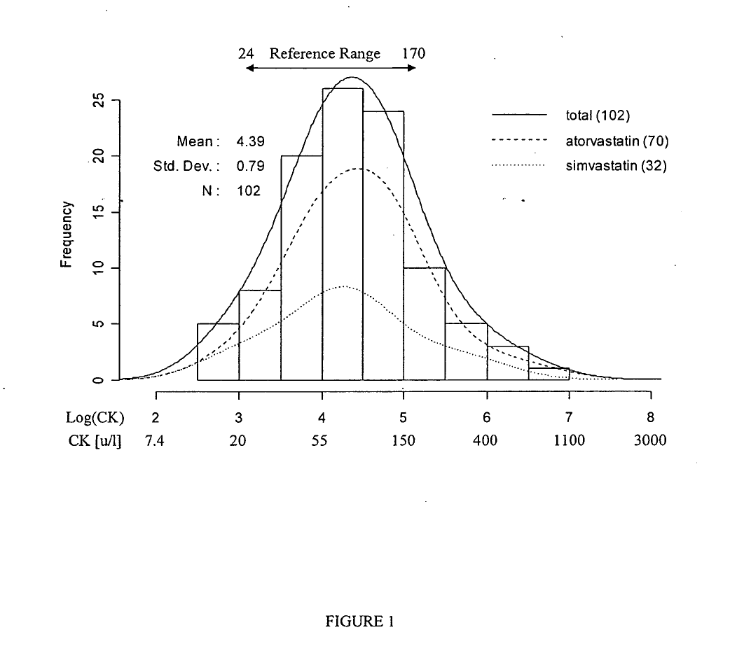

The distribution of log(CK) values in the study population was approximately normal (FIG. 1). A significant difference in CK between males and females was not observed, but the trend is as expected, with males somewhat higher (Table 1).

TABLE 1
Percentage count and mean CK activities (u/l) in the 102 patient
cohort organized by 3 demographic criteria and by statin therapy.

| Variable | Range | % Count | Median CK |
|---|---|---|---|
| Age | <40 | 3 | 73 |
| | 40-49 | 9 | 147 |
| | 50-59 | 23 | 105 |
| | 60-69 | 14 | 71 |
| | 70-79 | 32 | 86 |
| | ≧80 | 19 | 62 |
| Gender | male | 63 | 73 |
| | female | 37 | 102 |
| Ethnicity | Caucasian | 87 | 75 |
| | Hispanic | 9 | 101 |
| | African | 3 | 109 |
| | Asian | 1 | 162 |
| Statin | atorvastatin | 69 | 84 |
| | simvastatin | 31 | 77 |

Log(CK) values were significantly elevated with regards to the reference range (4.39 vs. 4.15, p<0.003 by one sample t-test), corresponding to a CK elevation of 27%. Fourteen out of 102 patients had elevated CK activities compared with the respective range limits of 140 u/l for females and 200 u/l for males. The significantly higher values for the study population as compared to normal indicated that a significant part of the CK is due to the effect of the drug.

The potential covariates of age, gender, race, statin, and dose were tested for association with log(CK) using multiple linear regression. Only age was found to be significantly associated. A decrease of log(CK) with age was observed, with a significance of p=0.0037, and explained 10% of the variation (R²=0.1). The coefficient was −0.02, meaning that each year of age lowers the expected log(CK) by 0.02, or CK activity by 2%.

TABLE 2

Assay information for the single nucleotide
polymorphisms (SNP's) in this study.

| | | | Alleles and Context |
|---|---|---|---|
| SNP | Gene | MAF* | Sequence on Assay Strand |
| rs2933249 | AGTR1 | 0.21 | TCCTTTGAATAATCAAACTGA[A/G] |
| | | | GAAGGAGAAGCAAGATGTCT |
| rs12695902 | AGTR1 | 0.10 | CATCAGGATTATCAGCATTTA[A/G] |
| | | | GCCAGAGTTGCAAATTAAGT |
| rs1549758 | NOS3 | 0.29 | TGGGTCCCCCCGCACAGAGCC[A/G] |
| | | | TCCTGCTGCCGGTAGCCCGC |
| rs1799983 | NOS3 | 0.26 | CAGAAGGAAGAGTTCTGGGGG[A/C] |
| | | | TCATCTGGGGCCTGCAGCAG |
| rs1800808 | SELP | 0.18 | ATGTGAATAATAAGGATAATA[A/G] |
| | | | TCACCAAATACATAGACATG |
| rs6136 | SELP | 0.10 | CAAGAGAATGGCCACTGGTCA[A/C] |
| | | | CTACCGTGCCAACCTGCCAA |
| rs6131 | SELP | 0.26 | GTCAGCACCTGGAAGCCCCCA[A/G] |
| | | | TGAAGGAACCATGGACTGTG |
| rs6092 | SERPINE1 | 0.12 | ACCTGCCTAGTCCTGGGCCTG[A/G] |
| | | | CCCTTGTCTTTGGTGAAGGG |
| rs5361 | SELE | 0.43 | GCCTGTACCAATACATCCTGC[A/C] |
| | | | GTGGCCACGGTGAATGTGTA |
| rs2742115 | OLR1 | 0.22 | ACATGTGTACACGTGGTGTAT[A/G] |
| | | | TTAAAAACTTCAGGCTCTCT |
| rs5369 | EDN1 | 0.15 | CACAAAGGCAACAGACCGTGA[A/G] |
| | | | AATAGATGCCAATGTGCTAG |
| rs877172 | OXT | 0.32 | GGTGAAGAGGCTGATGGGGCC[A/C] |
| | | | AGCAGGTCACAGAGCTCATC |
| rs1283718 | ANGPT1 | 0.08 | CTTCAAAAAGTGAAACTAACT[A/C] |
| | | | CTCGTTTCTGGTAAAGAGCC |
| rs2514869 | ANGPT1 | 0.14 | GGCAAAGTTTCATCTATTAGC[A/G] |
| | | | ATAAAATGTGAATTTTCTGC |
| rs1283694 | ANGPT1 | 0.20 | CAAACCCTTTCCACTCCATTA[A/T] |
| | | | AAGAACATGAATCCTGATAA |
| rs1570679 | FLT1 | 0.41 | CCCATGGCCACAACAGAACTC[A/G] |
| | | | CAAATGGCAGAGCTAGGGAG |
| rs2296189 | FLT1 | 0.18 | GATTTTGTCAAAGATAGATTC[A/G] |
| | | | GGAGCCATCCATTTCAGAGG |
| rs10507383 | FLT1 | 0.08 | CCCTTTCAGCAACAACACCAT[C/G] |
| | | | GGTAGAAATATGATGCAGCG |
| rs748253 | FLT1 | 0.41 | GCCCTGGTTTCCTCCAGTATG[A/C] |
| | | | CTGCAAAATTTCCTCTCCAT |

*minor allele frequency

Two genes, AGTR1 and NOS3, were found in the association tests as highly significantly associated with CK activity after false discovery rate (FDR) adjustment (Table 3).

TABLE 3
Genes studied with association p-values and FDR adjusted p-values

| LOCUS | P-value | FDR | Power | SNP type | Gene | Description |
|---|---|---|---|---|---|---|
| rs12695902 | 0.002 | 0.041 | 1.2 | intron 3 | AGTR1* | angiotensin II |
| | | | | | | receptor, type 1 |
| rs1799983 | 0.005 | 0.047 | 0.8 | D298E, | NOS3* | nitric oxide |
| | | | | exon 8 | | synthase 3 |
| | | | | | | (endothelial |
| | | | | | | cell) |
| rs2514869 | 0.016 | 0.10 | 1.0 | intron 8 | ANGPT1** | angiopoietin 1 |
| rs877172 | 0.022 | 0.11 | 0.8 | ˜2.3 kb | OXT | Oxytocin |
| | | | | upstream | | (Neurophysin 1) |
| rs2296189 | 0.037 | 0.14 | 0.9 | exon 24, | FLT1*** | fms-related |
| | | | | P1068P | | tyrosine kinase |
| | | | | | | 1 (vascular |
| | | | | | | endothelial growth |
| | | | | | | factor/vascular |
| | | | | | | permeability |
| | | | | | | factor receptor) |
| rs5369 | 0.19 | 0.45 | 1.0 | exon 3, | EDN1 | endothelin 1 |
| | | | | E106E | | |
| rs6136 | 0.46 | 0.79 | 1.2 | exon 13, | SELP** | selectin P |
| | | | | P756T | | (granule membrane |
| | | | | | | protein 140 kDa, |
| | | | | | | antigen CD62) |
| rs5361 | 0.59 | 0.93 | 0.7 | exon 3, | SELE | selectin E |
| | | | | R149S | | (endothelial |
| | | | | | | adhesion |
| | | | | | | molecule 1) |
| rs2742115 | 0.98 | 0.99 | 0.9 | intron 1 | OLR1 | oxidized low |
| | | | | | | density |
| | | | | | | lipoprotein |
| | | | | | | (lectin-like) |
| | | | | | | receptor 1 |
| rs6092 | 0.99 | 0.99 | 1.1 | exon 1, | SERPINE1 | serine (or |
| | | | | T15A | | cysteine) |
| | | | | | | proteinase |
| | | | | | | inhibitor, clade |
| | | | | | | E (nexin, |
| | | | | | | plasminogen |
| | | | | | | activator |
| | | | | | | inhibitor type 1), |
| | | | | | | member 1 |

Power is given as the effect size in standard deviations that can be detected at α = 0.05 with 80% power. Where there was more than one SNP per gene, only the most associated SNP is shown. The other SNPs are noted as follows:
*one less significant SNP not listed,
**two less significant SNPs not listed

Three other genes, ANGPT1, OXT, and FLT1 were by themselves statistically associated, but must be considered tentative because their FDR p-values imply a false discovery rate of 10-14% due to multiple testing. The remaining genes were not significantly associated.

The effect size detected based on the SNP frequency, sample size, and number of SNPs tested ranged between 0.7 and 1.2 standard deviations (Table 3). The standard deviation for log(CK) was 0.79 (see FIG. 1), which on the linear scale corresponded to a factor of 2.2.

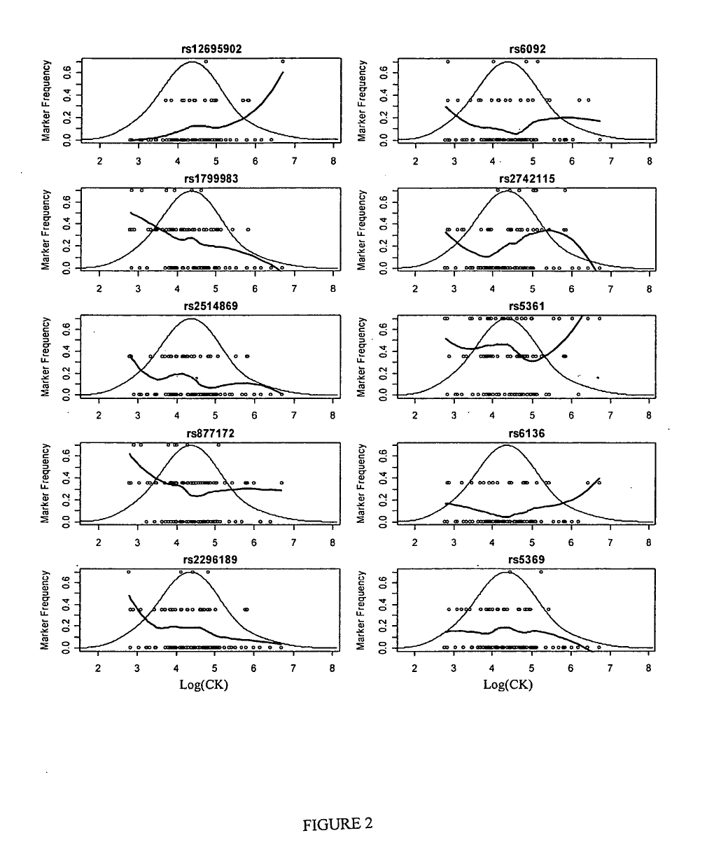

FIG. 2 shows a detailed representation of the genetic association tests for all 10 SNPs. The overall distribution of log(CK) is shown along with the individual genotypes and a LOESS fit of the allele frequency as a function of log(CK). The bell curve shows the actual distribution of CK activity phenotype in the clinical database. The LOESS curve shows the localized frequency of the least common allele for sectors of the distribution. For SNPs with a strong association, the marker frequency is significantly different between the high end and the low end of the distribution. Conversely, if a marker is neutral, the frequency is independent on the CK activity and the LOESS curve is essentially flat. For example, the first figure of the panel shows the LOESS curve for SNP rs12695902 which is located in the gene for angiotensin II receptor, type 1 (AGTR1). The frequency of the minor allele is almost zero in subjects with low CK activity, whereas it approaches 50% at the high end of CK activity. This is indicative of a strong association between the marker and CK activity.

### Discussion

The metabolic mediators of statin myopathy are unknown. In the present report, physiogenomic analysis were used to examine the relationship between genes affecting vascular function and serum CK activity in statin users. In this approach, genetic associations to a phenotype were used to suggest possible physiological mechanisms underlying it. The results suggested that genetic variants in the AGTR1 and NOS3 genes were very significantly associated with CK activity in patients treated with statins.

The endothelium regulates vascular tone through the release of vasoactive substances (Rubanyi, 1991). One of the most important vasocontrictors is angiotensin II, which stimulates a variety of pro-atherogenic responses, such as expression of adhesion molecules, platelet aggregation, thrombosis and cell migration. Its receptor, AGTR1, was included in the survey and found it to evidence the most significant genetic association to serum CK activity.

The most important vasodilator is NO, generated by the endothelial nitric oxide synthase (NOS3) (Zöllner et al 1997). NO also inhibits inflammation, oxidation, vascular smooth muscle cell proliferation, and migration. NOS3 was the second ranking gene in our survey, and also very significantly associated with serum CK activity.

Three genes evidenced a weaker statistical association. Angiopoietin-1 was the third ranking gene in the survey. This hormone has been shown to counteract cell death by apoptosis in cultured endothelial cells (Holash et al, Kwak et al). Oxytocin and FMT-related tyrosine kinase 1, ranked fourth and fifth in our survey. Genes surveyed but without any significant associations included endothelin-1, selectins P and E, oxidized low-density lipoprotein receptor, and SERPINE-1.

The physiogenomics approach does not require a conventional control composed of untreated or placebo cohorts (See also, related U.S. patent application Ser. Nos. 10/868,863 and 11/010,716, the contents of each of which are incorporated by reference in their entirety). The distribution of patients similarly treated with statins in effect establishes the comparison groups between patients below the mean response versus patients above. It can be surmised that CK activity is related to the associated genes during statin therapy because of the observed elevated CK for the cohort as a whole.

Heretofore, most of the muscular effects of statins have been ascribed to skeletal muscle, prompted by clinical manifestations of myalgia, and by histopathology of muscle biopsies. However, vascular smooth muscle has pervasive exposure to a circulating statin given its close apposition to the endothelium. The present results related CK activity during statin therapy to genes affecting vascular smooth muscle and raise the novel hypothesis that statins affect smooth muscle via alterations in vascular function.

### EXAMPLE 2

PHYSIOGENOMICS ARRAY A gene array covering 384 SNPs corresponding to 214 genes related to six major physiological axes: cardiovascular function, inflammation, neurobiology, metabolism, lipid biochemistry, and cell growth was developed. The following pathways were represented: insulin resistance, glucose metabolism, energy homeostasis, adiposity, apolipoproteins and receptors, fatty acid and cholesterol metabolism, lipases, receptors, cell signaling and transcriptional regulation, growth factors, drug metabolism, blood pressure, vascular signaling, endothelial dysfunction, coagulation and fibrinolysis, vascular inflammation, cytokines, neurotransmitter axes (serotonin, dopamine: cholinergic, histamine, glutamate) and behavior (satiety). The array has been used successfully on approximately 1000 samples from different clinical studies. The array was assembled using the methods described herein and genotyping on the array was performed on 96 samples each using the Illumina BeadArray® genotyping platform. A listing for genes in the Physiogenomics Array is shown in Table 4.

TABLE 4
Physiogenomics Array Gene Array.

Metabolic Response and Insulin Resistance
Insulin Resistance: insulin, insulin receptor, insulin
receptor substrate 1, Akt1, Akt2, cholecystokinin and
cholecystokinin receptor A and B, resistin, regulatory
subunit of PI3-kinase (polypeptide 1, p85α), ATP-binding
cassette B1, C8, and G5
Glucose Metabolism: glucagon, glycogen synthase 1, 2, 3β,
phosphofructokinase (liver, muscle, platelet), pyruvate
kinase (liver, RBC, muscle), phosphoenolpyruvate
carboxykinase 1
Energy Homeostasis: uncoupling protein 2 and 3, adrenergic
receptor α1A, α2A, α2B, β1, β2, β3, carnitine palmitoyl-
transferase 1A, 1B, 2, pro-melanin-concentrating hormone,
melanocortin receptor 3 and 4, pro-opiomelanocortin, malate
dehydrogenase, AMP kinase, subunit α1, catalytic AMP-
activated protein kinase α-2, non-catalytic AMP-activated
protein kinase β1, β2, γ1, 2, 3
Adiposity: leptin receptor, ghrelin precursor, adiponectin
receptor 1 and 2, adipocyte, C1Q and collagen domain
Other: glucocorticoid receptor, corticotropin releasing
hormone and ˜receptor 1 and 2, organic anion transporter,
carbohydrate response element-binding protein
Cholesterol Homeostasis and Biosynthesis
Apolipoproteins: apolipoprotein A1, A2, A4, A5, B, C1 to C4,
D, E, F, H, L-I to L-V, and M
Fatty Acid and Cholesterol Metabolism: acetyl-CoA-acetyl-
transferase 1, 2, acetyl-CoA carboxylase α, β, CETP, LCAT,
fatty acid synthase, fatty acid-binding protein 2, micro-
somal triglyceride transfer protein, paraoxonase 1
Lipases: hepatic, lipoprotein, hormone-sensitive, lysosomal
acid, endothelial, and gastric
Receptors: scavenger receptor B1 and B2, LDL receptor
Growth factors: BNDF, insulin-like growth factor 1, growth
hormone 1, growth hormone releasing hormone
Drug Metabolism: cytochrome P450 1A2, 2C19, 2D6, subfamily
IIIA, polypeptide 4 and 5, cytochrome P-450 type 7
Cell Signaling (A) and Transcriptional Regulation (B): (A)
catalytic PI3K α, β, γ, δ polypeptide, class 2 PI3K β, γ
polypeptide, class 3 PI3K, catalytic PI3K, α polypeptide,
(B) peroxisome proliferation-activated receptor α, γ,
hypoxia-inducible factor 1A, sterol regulatory element
binding transcription factor 1, retinoic receptor α, β, γ,
retinoid X receptor α, β, γ
Cardiovascular Function
Blood Pressure: endothelial nitric oxide synthase,
angiotensin I converting enzyme 1, endothelial type 1 and 2
angiotensin II receptor, angiotensinogen, endothelin-1,
oxytocin, phenylethanolamine N-methyltransferase
Vascular Signaling: vascular endothelial growth factor A,
VEGF receptors (KDR, FRT1), angiopoietin 1, 2, TEK tyrosine
kinase
Endothelial Dysfunction: oxidized low-density lipoprotein
receptor, Cu/Zn-superoxide dismutase
Coagulation/Fibrinolysis: thrombin and receptor, E-selectin,
P-selectin, plasminogen activator inhibitor 1, GNOA
Other (e.g., cardiovascular disease related): 5,10-
methylenetetrahydrofolate reductase, cardiac-ankyrin repeat
protein
Vascular Inflammation
Vascular Inflammation: vascular cell adhesion molecule 1,
intercellular adhesion molecule 1 (CD54), platelet/
endothelial cell adhesion molecule (CD31 antigen),
C-reactive protein, chemokine ligand 2, amiloride binding
protein 1
Cytokines: interleukin 1α and 1β, 2, 6, 10, Interleukin
Receptor, TNF (ligand) superfamily, member 1A, 1B, 2, 6,
α-induced TNF protein 6, β-transforming growth factor
Neurotransmission
Serotonin: 5α-hydroxytryptamine receptor 1A, 1D, 2A, 2C,
3A, 3B, 5A, 6, 7, serotonin transporter
Dopamine: COMT, dopamine receptor D1 interacting protein,
receptor D1 to D5, transporter, decarboxylase,
β-hydroxylase, tyrosine hydroxylase
Cholinergic: choline acetyltransferase, acetylcholinester-
ase, muscarinic cholinergic receptor 1, 2, 3, 5, neuronal
nicotinic cholinergic receptor, alpha polypeptide 7,
galanin
Histamine: N-methyltransferase, receptor H1 to H3
Glutamate: GABA receptor α2, α4, glutamate decarboxylase 1
and 2, D-amino-acid oxidase, ornithine amino transferase
Behavior (Satiety, obesity): cocaine- and amphetamine
regulated transcript, hypocretin, neuropeptide Y,
neuropeptide Y receptor Y1, Y5, peptide YY, somatostatin
and receptor 3, 5
Psychoatric Disorder related: drosophila homolog of NOTCH
4, disrupted in schizophrenia 1 (DISC1), dystrobrevin-
binding protein dysbindin

Clinical Database and Repository. An existing clinical database and DNA repository of statin treated patients assembled by Dr. Alan Wu at Hartford Hospital was utilized. Medical records of adult patients (>21 y) at the Hartford Hospital Lipid Clinic were examined to determine eligibility. Patients were included if they had one of four statins (lovastatin, simvastatin, atorvastatin, and pravastatin) prescribed, understood the protocol, and signed an informed consent. One EDTA-preserved (lavender top tube) blood sample (4 ml) was collected for SNP analysis. Although 288 patients were recruited, good genotype data is available for 134 patients and 324 SNPs. The patients were clinically characterized with measured serum creatine kinase levels, their statin exposure, and questions regarding their experience of myalgia. Subjects were considered to have developed statin related myalgias if they reported new or increased myalgia, cramps, or muscle ache after statin administration and if the symptoms persisted for at least 2 weeks of statin administration. Among the 288 patients, 196 were classified as free of statin induced myalgia (“no”), while 78 were classified as symptomatic (“yes”).

Associations discovered with the Physiogenomics Array and the Clinical Database. Samples were screened for SNPs associated with the observation of myopathy (as assessed by myalgia and elevated CK) in patients treated with statin drugs. The endpoints analyzed were measured serum creatine kinase and the development of myalgia. For the myalgia endpoint, the classification was converted into a numeric index, with the assignments “no”->0, “maybe”->0.5, and “yes”->1. Table 5 shows the analysis of variance for the baseline models (non-genetic) for serum creatine kinase and the myalgia index. The only significantly predictive covariate was age, and the baseline models explain only 4% of the variation in each response variable, leaving most of the variation potentially explained by genetic markers. There was no significant association with statin type, suggesting that any detected myopathic effect was independent of which statin has been prescribed.

TABLE 5
Baseline models for creatine kinase and myalgia index.

| Response | Predictor Variable | Explains | P-value |
|---|---|---|---|
| Serum Creatine | Age | 0% | 0.449 |
| Kinase (log | Gender | 1% | 0.188 |
| concentration) | Statin | 3% | 0.234 |
| | Dose | 0% | 0.838 |
| | Total | 4% | |
| Myalgia Index (no: 0, | | | |
| maybe: 0.5, yes: 1) | Age | 3% | 0.016 |
| | Gender | 0% | 0.835 |
| | Statin | 1% | 0.622 |
| | Dose | 0% | 0.877 |
| | Total | 4% | |

Table 6 shows the results of the SNP association screen. The p-values for each SNP were obtained by adding the SNP to the baseline model and comparing the resulting model improvement with up to 10,000 simulated model improvements using the same data set, but with the genotype data randomly permuted to remove any true association. This method produced a p-value that was a direct, unbiased, and model-free estimate of the probability of finding a model as good as the one tested when the null hypothesis of no association was true.

TABLE 6
Results of SNP screen for creatine kinase and myalgia index

| Response | Locus | P-value | Gene | Description |
|---|---|---|---|---|
| Serum Creatine | rs1799983 | 0.000897 | NOS3 | nitric oxide synthase 3 (endothelial |
| Kinase (log | | | | cell) |
| concentration) | rs877172 | 0.002089 | OXT | Oxytocin (Neurophysin 1) |
| | rs675 | 0.003188 | APOA4 | apolipoprotein A-IV |
| | rs12695902 | 0.003272 | AGTR1 | angiotensin II receptor, type 1 |
| | rs2740574 | 0.0053 | CYP3A4 | cytochrome P450, family 3, subfamily |
| | | | | A, polypeptide 4 |
| | rs1800716 | 0.0059 | CYP2D6 | CYP2D6 cytochrome P450, family 2, |
| | | | | subfamily D, polypeptide 6 |
| | rs2020933 | 0.012419 | SLC6A4 | solute carrier family 6 (neurotransmitter |
| | | | | transporter, serotonin), member 4 |
| | rs2296189 | 0.013912 | FLT1 | fms-related tyrosine kinase 1 (vascular |
| | | | | endothelial growth factor/vascular |
| | | | | permeability factor receptor) |
| | rs2070424 | 0.013934 | SOD1 | superoxide dismutase 1, soluble |
| | | | | (amyotrophic lateral sclerosis 1 (adult)) |
| | rs854572 | 0.01436 | PON1 | paraoxonase 1 |
| | rs3756450 | 0.01677 | SLC6A3 | solute carrier family 6 (neurotransmitter |
| | | | | transporter, dopamine), member 3 |
| | rs1611115 | 0.024944 | DBH | dopamine beta-hydroxylase (dopamine |
| | | | | beta-monooxygenase) |
| | rs600728 | 0.034215 | TEK | TEK tyrosine kinase, endothelial |
| | | | | (venous malformations, multiple |
| | | | | cutaneous and mucosal) |
| | rs3176921 | 0.035228 | CRH | corticotropin releasing hormone |
| | rs10841044 | 0.037906 | PIK3C2G | phosphoinositide-3-kinase, class 2, |
| | | | | gamma polypeptide |
| | rs7200210 | 0.038947 | SLC12A4 | solute carrier family 12 |
| | | | | (potassium/chloride transporters), |
| | | | | member 4 |
| | rs5491 | 0.039004 | ICAM1 | intercellular adhesion molecule 1 |
| | | | | (CD54), human rhinovirus receptor |
| | rs617333 | 0.041033 | TEK | TEK tyrosine kinase, endothelial |
| | | | | (venous malformations, multiple |
| | | | | cutaneous and mucosal) |
| | rs1549758 | 0.046919 | NOS3 | nitric oxide synthase 3 (endothelial |
| | | | | cell) |
| | rs2514869 | 0.049412 | ANGPT1 | angiopoietin 1 |
| Myalgia Index | rs2058112 | 0.004 | ADIPOR2 | adiponectin receptor 2 |
| | rs1800794 | 0.006121 | IL1A | interleukin 1, alpha |
| | rs504714 | 0.0098 | AVEN | apoptosis, caspase activation inhibitor |
| | rs6195 | 0.012992 | NR3C1 | nuclear receptor subfamily 3, group C, |
| | | | | member 1 (glucocorticoid receptor) |
| | rs2742115 | 0.014493 | OLR1 | oxidized low density lipoprotein (lectin- |
| | | | | like) receptor 1 |
| | rs1042718 | 0.022686 | ADRB2 | adrenergic, beta-2-, receptor, surface |
| | rs2276307 | 0.025031 | HTR3B | 5-hydroxytryptamine (serotonin) |
| | | | | receptor 3B |
| | rs7412 | 0.02927 | APOE | apolipoprotein E |
| | rs6488950 | 0.030137 | SCARB1 | scavenger receptor class B, member 1 |
| | rs9904270 | 0.031869 | RARA | retinoic acid receptor, alpha |
| | rs1570679 | 0.033483 | FLT1 | fms-related tyrosine kinase 1 (vascular |
| | | | | endothelial growth factor/vascular |
| | | | | permeability factor receptor) |
| | rs2049045 | 0.038544 | BDNF | brain-derived neurotrophic factor |
| | rs6265 | 0.039108 | BDNF | brain-derived neurotrophic factor |
| | rs132653 | 0.044448 | APOL3 | apolipoprotein L, 3 |
| | rs6318 | 0.047214 | HTR2C | 5-hydroxytryptamine (serotonin) |
| | | | | receptor 2C |
| | rs2838549 | 0.048431 | PFKL | phosphofructokinase, liver |

This genetic screen yielded associations for CK elevation and myalgia. The CYP3A4 isoenzyme was the main metabolic substrate for atorvastatin and simvastatin. FLT1 (also known as fms-related tyrosine kinase 1 and vascular endothelial growth factor/vascular permeability factor receptor) was specifically expressed in most of the vascular endothelial cells. AdipoR2, one of the two recently identified receptors for adiponectin, an adipocyte-specific secreted hormone with anti-diabetic and anti-atherogenic activities, was expressed in skeletal muscle and human atherosclerotic lesions (Chinetti et al. 2004). These results supported the basic physiogenomic approach as a novel means of identifying genetic markers, and that the Physiogenomics Array applied to our clinical database and repository was a fundamental resource for the process.

### EXAMPLE 3

### Selection of Gene Markers for SIM Gene Array

Several theories exist on the general blockage of cholesterol synthesis, the reduction in local ubiquinone levels or the interference with signaling cascades leading to apoptosis. A multitude of candidate genes exists from the known action of statins on lipid metabolism and skeletal muscle physiology and can form the basis for a specialized gene array for statin injury to muscle (SIM) and muscle side effects.

In the selection of candidate genes representatives of various physiological pathways and networks is utilized (table 7). The genes included represent the primary therapeutic targets of statins and their pharmacological pathways, the known and potential downstream targets of statins as part of the cholesterol and lipid metabolism pathways, and the known and hypothesized genetic risk factors for the development of myopathies. Although the list of genes most likely misses some known key genes and lacks as of yet undiscovered, the built-in redundancy, feedback, and amplification of many networks indicates that the elucidation of every single gene in a pathway is likely unnecessary for physiogenomics.

A SIM Gene Array is built using the compiled a list of 200 candidate genes representing key pathways in statin metabolism, cholesterol and lipid homeostasis, energy efficiency, including ubiquinone and mitochondrial pathways, muscle maintenance and repair, including apoptosis and inflammation, as well as muscle contraction (table 7). The genetic association analysis of those genes offers an opportunity to develop predictive tools for the identification of patients at risk to develop statin-induced side effects and will also provide a powerful alternative to better understand their molecular basis. Detailed motivations for the gene selection are described in the following paragraphs.

TABLE 7
Selection of Candidate Genes for the SIM Gene Array. Genes are drawn from physiological pathways.

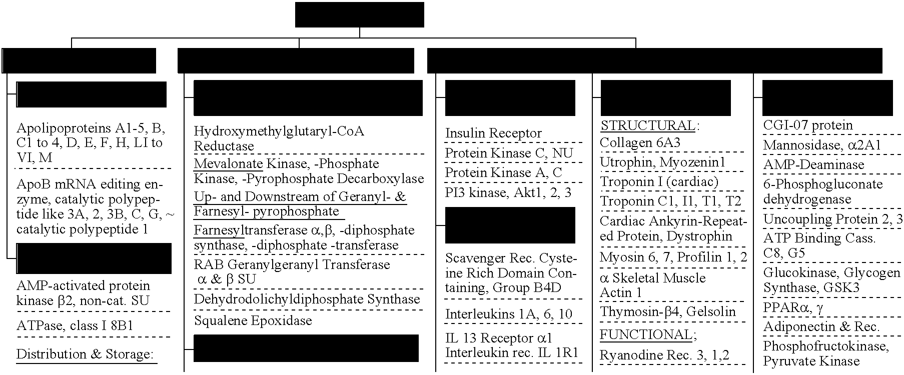

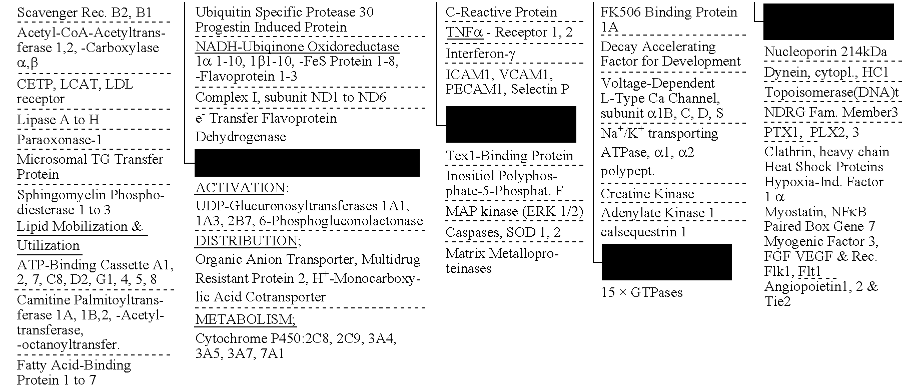

a. Primary and Secondary Therapeutic Targets of Statins and Pharmacological Pathways

1. Primary and Secondary Targets of Statins. The primary target of statins is the HMG-CoA reductase, the first and rate-limiting step in cholesterol biosynthesis. One theory about the molecular basis of statin-induced myalgias maintains despite controversy that blocking cholesterol synthesis reduces the cholesterol content of skeletal muscle cell membranes, making the membranes unstable. More importantly, downstream products of HMG-CoA reductase are cholesterol precursors. These are important for several cell functions and serve, for example, glycosylation of cell surface proteins, electron transfer during mitochondrial respiration, and post-translational modification of regulatory proteins (Nakagami et al. 2003).

2. Ubiquinone Pathway. Alternatively, statins reduce the production of isoprenoids, such as ubiquinone. Ubiquinone, or Coenzyme Q10, participates in the electron transport during oxidative phosphorylation in mammalian mitochondria (Crane 2001). Genetic variations of ubiquinone-associated mitochondrial genes can reduce the efficacy of the electron transport. For example, a 7-bp inversion change in the gene for the subunit ND1 of complex I was found to be causative for mitochondrial myopathy with isolated complex I deficiency (Musumeci et al. 2000). Serum ubiquinone levels decrease with statin treatment probably because ubiquinone is transported in the LDL particle (Ghirlanda et al. 1994). Intramuscular ubiquinone levels do not decrease, however (Laaksonen et al. 1994).

3. Statin Pharmacology. All statins except pravastatin are metabolized through the hepatic cytochrome P450 (CYP) enzyme system (phase I), and specific hepatic cytochrome isoenzymes have been identified for each drug (Schmitz, Drobnik 2003). The CYP3A4 isoenzyme is responsible for lovastatin, simvastatin, and atorvastatin, while CYP2C9 is responsible for fluvastatin. CYP3A4 accounts for >50% of the total hepatic P450 activity. The statins are also metabolized through acyl-glucuronidation (phase II) (Dimitroulakos, Yeger 1996), and through biliary secretion through the assistance of P-glycoprotein, a protein that is co-localized with CYP3A4 (Hsiang et al. 1999, Yamazaki et al. 1997). Encoded by multiple drug resistance (MDR)-gene, P-glycoprotein functions as an exporter of drugs and chemicals into the bile and urine for excretion. Uptake of these drugs into the liver occurs with the assistance of organic anion transporting polypeptide (OATP). The C-isoform is liver-specific and supports the membrane translocation of bile acids, peptides, and sulfated conjugates. Single nucleotide polymorphisms exist in both the cytochrome P450 isoenzyme system and the MDR1 gene. For CYP, polymorphisms are associated with tremendous variability in the rate of metabolism (e.g., >40-fold for CYP3A4 (Wolf, Smith 1999)). Clinical laboratories are beginning to routinely monitor CYP2C9 polymorphism to predict patient response to warfarin therapy (Linder et al. 2002). Polymorphisms in the CYP3A4 gene have been described in the 5′ promoter region where there is a A to G substitution in codon −290 (von Ahsen et al. 2001). The frequency of this allele is quite variable from 0% for Asians, 10% for Caucasians, and 55% for African Americans (Ball et al. 1999). The most widely studied polymorphism of MDR1 occurs at nucleotide position 3435 where there is a C to T transition. The heterozygote allele frequency is roughly 50% in Caucasians and is considerably less in African Americans (Yates et al. 2003). Presence of the CC wildtype is associated with a two-fold higher P-glycoprotein expression TT genotype (Fromm 2002). There are over a dozen SNPs in the OAPT-C gene. A recent study showed that SNPs at nucleotide positions 388 and 521 affect the pharmacokinetics of pravastatin (Nishizato et al. 2003). The allele frequencies of the 388 polymorphism is 30% in European Americans, 60% in Japanese, and 75% in African Americans. The corresponding frequencies for the SNP at 521 are 16, 14, and 2%, respectively. The OAPT-C SNP at 521 has been associated with a decreased substrate transport activity (Tirona, Kim 2002) and may be a target for statin metabolism.

b. Cholesterol and Lipid Metabolism

1. Apolipoproteins. Apolipoproteins (Apo), the structural components of lipoproteins are being studied in CVD for their role in atherosclerotic plaque development (Boden 2000, Ribalta et al. 2003). They assist in the transport of cholesterol from bodily tissues to the liver for excretion (ApoA1) and in the transport and conversion of triglycerides (ApoB). Apolipoproteins are also involved in the metabolism of triglyceride-rich lipoproteins (ApoE) and represent cofactors for lipid modifying proteins (ApoA1 for lecithin:cholesterol acyltransferase, ApoC for lipoprotein lipase).

2. Lipid Metabolism. From a variety of regulatory enzymes of glycolysis and lipogenesis of interest, pyruvate kinase, phosphofructokinase, acetyl CoA carboxylase, and fatty acid synthase are included among others. Lipoprotein lipase plays an important role in VLDL fatty acid release and its subsequent conversion to LDL. Hormone-sensitive lipase is a major determinant of fatty acid mobilization. It plays a pivotal role in lipid metabolism, overall energy homeostasis, and fatty acid signaling. Since hepatic lipase can have a function in the metabolism of both pro- and anti-atherogenic lipoproteins, it denotes another interesting gene target. Two proteins have been selected as part of the free fatty acid metabolism (Oakes and Furler 2002). Carnitine palmitoyltransferase (CPT) facilitates mitochondrial fatty acid oxidation and deficiencies in CPT are common disorders. The intestinal fatty acid binding protein gene is of further interest since it has been proposed as a candidate gene for diabetes. For controlling amounts of fatty acids, cells are endowed with two acetyl-coenzyme A carboxylase (ACC) systems. In particular, ACCB is believed to control mitochondrial fatty acid oxidation. ATP-binding cassette (ABC) transporters modulate cholesterol and lipoprotein metabolism (Ribalta et al. 2003). ABCG5 and ABCG8 play an important role in limiting intestinal absorption and promoting biliary excretion of neutral sterols.

c. Potential Targets of Statin Side Effects in Skeletal Muscles

1. Metabolism and Energy Efficiency. Insufficient supply with metabolites and energy will affect the muscle primarily when challenged. Glycogen synthase activity is thought to be rate-limiting in the disposal of glucose as muscle glycogen (Nielsen and Richter 2003). The enzyme is regulated by post-transcriptional phosphorylation through the phosphoinositol-3 kinase (PI3K) pathway, making it a response element for growth factor signaling. Phosphoenolpyruvate carboxykinase is considered to be the first step in gluconeogenesis. The synthesis of the soluble isoform is regulated by gene transcription and the rate of mRNA turnover can be induced by starvation and reduced through a high carbohydrate diet. Adiponectin and resistin are secretory products of adipose tissue (Beltowski 2003). Adiponectin stimulates fatty acid oxidation, decreases plasma triglycerides, and improves glucose metabolism by increasing insulin sensitivity. It inhibits inflammation and atherogenesis by suppressing the migration of monocytes and their transformation into foam cells. Plasma adiponectin is reduced in MetSyn and in patients with ischemic heart disease. Hypoadiponectinemia may contribute to insulin resistance and accelerated atherogenesis in obesity. The role of resistin in linking human obesity with diabetes 2 is indicated but still questionable. Uncoupling proteins UCP2 and UCP3 play a role in reducing reactive oxygen species formation (Giacobirno 2001). UCP3 could also facilitate lipid oxidation by acting as a free fatty acid anion transporter in a variety of physiological states. A cornerstone achievement was the correlation of statin-induced myopathy to “ragged red fibers” in muscle specimen examined microscopically (Phillips et al. 2002). Genetic defects of mitochondrial genes in the respiratory chain have been associated with this morphology and excessive accumulation of mitochondria in the muscle.

2. Recovery and Maintenance (Including Inflammation and Apoptosis). The mechanical challenges muscles have to face are being met in the healthy organism by continuous maintenance and repair mechanisms. The inhibition of those processes not only slows down the recovery to pre-challenge conditions but can also lead to accumulation of toxic metabolites. Factors involved in muscle gene transcription and translation play a key role in the recovery process. The downregulation of transcription factors, such as paired-like homeodomain transcription factor 1 (Table 4), is important for development of the hindlimbs and represents a strong lead for a potential mechanism of statin-induced myopathies. Other transcription factors that have been discussed as potential mediators are paired box genes, NFκB, and hypoxia-inducible factor 1α. Growth and differentiation factors, such as fibroblast growth factor, myostatin, or myogenic factor 3 encompass another category. Gonzalez-Cadavid et al. (1998) examined the hypothesis that myostatin expression correlates inversely with fat-free mass in humans and that increased expression of the myostatin gene is associated with weight loss in men with AIDS wasting syndrome. Myogenic factor 3 regulates skeletal muscle differentiation and is essential for repair of damaged tissue. NFκB is activated by the cytokine tumor necrosis factor (TNF), a mediator of skeletal muscle wasting in cachexia (Guttridge et al. 2000). TNF-induced activation of NFκB has been shown to inhibit smooth skeletal muscle differentiation by suppressing myogenic factor 3 mRNA at the posttranscriptional level. In contrast, in differentiated myotubes, TNF plus interferon-γ signaling was required for NFκB-dependent downregulation of myogenic factor 3 and dysfunction of skeletal myofibers. Statins in general seem to diminish systemic inflammation as a substantial component of the atherosclerotic process. Both fenofibrate and simvastatin were shown to markedly reduce plasma levels of high-sensitivity C-reactive protein (CRP), IL-1β, and CD40L (tumor necrosis factor ligand SF5), and to improved endothelium-dependent flow-mediated dilation of the brachial artery (Wang et al. 2003). Recently, statins have been shown to inhibit cardiac hypertrophy and provide cardioprotection, possibly attributed to their functional influences on small G proteins such as Ras and Rho. The blocking of isoprenylation, results in an increase of endogenous nitric oxide, reduction of oxidative stress, inhibition of inflammatory reaction, and decrease of the renin-angiotensin system activity as well as C-reactive protein levels in cardiac tissues (Auer et al. 2002, Nakagami et al. 2003). The statin induced downregulation of GTPases observed in healthy subjects is remarkable and will be further addressed by carefully selecting genes of this category.

3. Muscle Contraction. Genes encoding structural and functional proteins involved in all aspects of muscle contraction represent an additional important category of potential targets for our physiogenomic approach to identify markers for susceptibility to statin-induced myalgias. The gene expression analysis showed clearly that statin treatment challenges the muscle by downregulating a series of structural proteins. Mutations of the respective genes have been linked to muscle-related diseases: collagen and utrophin mutations associate with different diagnosis of myopathies and dystrophies, myozenin is discussed as a candidate gene for limb-girdle muscular dystrophy and other neuromuscular disorders, and troponin modifications are responsible for familial hypertrophic cardiomyopathy. Functional aspects of muscle physiology are predominantly related to calcium transport, storage, in addition to energy supply of the muscle. The ryanodine receptor on the sarcoplasmic reticulum is the major source of calcium required for muscle excitation-contraction coupling. The channel is comprised of ryanodine receptor polypeptides and FK506-binding proteins, both differentially regulated in muscle biopsies of statin treated subjects (Table 4). Protein kinase A phosphorylation of the ryanodine receptor polypeptide dissociates the FK506-binding protein and regulates the channel open probability.

4. Neurotransmission. Genes encoding proteins involved in all aspects of neurotransmission are included, to detect effects related to pain perception, activity levels, and other neurological aspects relevant to the study of muscle pain. The following is a categorized list of such genes that are considered include, but are not limited to: Serotonin: 5α-hydroxytryptamine receptor 1A, 1D, 2A, 2C, 3A, 3B, 5A, 6, 7, serotonin transporter. Dopamine: COMT, dopamine receptor D1 interacting protein, ˜receptor D1 to D5, ˜transporter, ˜decarboxylase, ˜β-hydroxylase, tyrosine hydroxylase. Cholinergic: choline acetyltransferase, acetylcholinesterase, muscarinic cholinergic receptor 1, 2, 3, 5, neuronal nicotinic cholinergic receptor, alpha polypeptide 7, galanin. Histamine: ˜N-methyltransferase, ˜receptor H1 to H3. Glutamate: GABA receptor α2, α4, glutamate decarboxylase 1 and 2, D-amino-acid oxidase, ornithine amino transferase. Behavior: cocaine- and amphetamine regulated transcript, hypocretin, neuropeptide Y, neuropeptide Y receptor Y1, Y5, peptide YY, somatostatin and ˜receptor 3, 5. Psychiatric Disorder related: drosophila homolog of NOTCH 4, disrupted in schizophrenia 1 (DISC1), dystrobrevin-binding protein dysbindin.

SIM Gene Array. Proprietary resources provide specific values for the selection of SNPs: the SNP Validation Code and the SNP Score. The SNP Validation Code describes the level of confirmation of the particular SNP (e.g., confirmed in HapMap project, confirmation by frequency and cluster, or low if SNP is not already confirmed). Public databases (dbSNP, ensembl) are searched for validated SNPs with known heterozygosities (HET) for mixed or Caucasian populations.

The low HET limit is set to 10% to ensure a sufficient representation of the respective SNP. The high HET limit was set at 30% under the assumption that alleles with a close to even distribution are more likely to be neutral (no phenotype). The number of SNPs per gene was based on the length of the gene: <25 kb=1 SNP, 25 to 100 kb=2 SNPs, >100 kb=3 SNPs.

### Statistical Plan

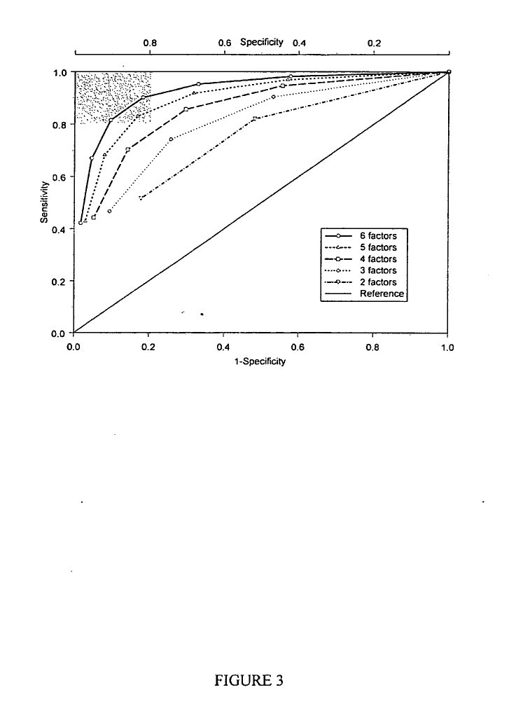

a. Data analysis. The objective of the statistical analysis is to find a set of physiogenomic factors that together provide a way of predicting the outcome of interest, in this case the occurrence of myalgia in a population of subjects. The association of an individual factor with the outcome may not have sufficient discrimination ability to provide the necessary sensitivity and specificity, but by combining the effect of several such factors the objective is reached. FIG. 3 shows a receiver operator characteristic (ROC) curve that demonstrates the relationship between sensitivity and specificity for the cumulative effect on prediction that can be achieved through the use of common factors that are statistically independent. The assumptions on which these calculations are based are (a) the factors are independent of each other, (b) the association between each factor and the outcome can be summarized by a modest odds ratio of 1.7, and (c) the prevalence of each physiogenomic factor in the population is 50% and independent of the others. Clearly, the prediction becomes even stronger if the association with the response is stronger or one finds additional predictors. However, factors that are less useful for these types of prediction are those that are less common in the population, or collinear with factors that have already been identified in the prediction model.

b. Model Building. Once the associated markers are determined, a model is developed for the purpose of predicting a given response, in this case the development of SIM. A linear logistic model will be used which can be expressed as follows:

$$logit ( Pr \{ Myalgia \text{\textbar} M , D \} ) = {R}_{0} + {\sum }_{i} {α}_{i} {M}_{i} + {\sum }_{j} {β}_{j} {D}_{j}$$

where Mi are the marker variables and Dj are demographic covariates. The model parameters that are estimated from the data are R₀, and, which is accomplished through the use of a generalized linear model in order to obtain maximum likelihood estimates of the parameters. (McCullagh and Nelder 1989). S-plus provides very good support for algorithms that provide these estimates for the initial linear regression models: as well as other generalized linear models that are used when the error distribution is not normal. For continuous variables, generalized additive models are considered (Hastie and Tibshirani 1986), including cubic splines (Durrleman and Simon 1989) in order to appropriately assess the form for the dose-response relationship.

In addition to optimizing the parameters, model refinement is performed. The first phase of the regression analysis will consist of considering a set of simplified models by eliminating each variable in turn and re-optimizing the likelihood function. The ratio between the two maximum likelihoods of the original vs. the simplified model then provides a significance measure for the contribution of each variable to the model.

The association between each physiogenomic factor and the outcome is calculated using logistic regression models, controlling for the other factors that have been found to be relevant. The magnitude of these associations are measured with the odds ratio and the corresponding 95% confidence interval, and statistical significance assessed using a likelihood ratio test. Multivariate analyses is used which includes all factors that have been found to be important based on univariate analyses.

Because the number of possible comparisons can become very large in analyses that evaluate the combined effects of two or more genes, the results include a random permutation test for the null hypothesis of no effect for two through five combinations of genes. This is accomplished by randomly assigning the outcome to each individual in the study, which is implied by the null distribution of no genetic effect, and estimating the test statistic that corresponds to the null hypothesis of the gene combination effect. Repeating this process 1000 times will provide an empirical estimate of the distribution for the test statistic, and hence a p-value that takes into account the process that gave rise to the multiple comparisons. In addition, hierarchical regression analysis is considered to generate estimates incorporating prior information about the biological activity of the gene variants. In this type of analysis, multiple genotypes and other risk factors can be considered simultaneously as a set, and estimates will be adjusted based on prior information and the observed covariance, theoretically improving the accuracy and precision of effect estimates (Steenland et al. 2000).

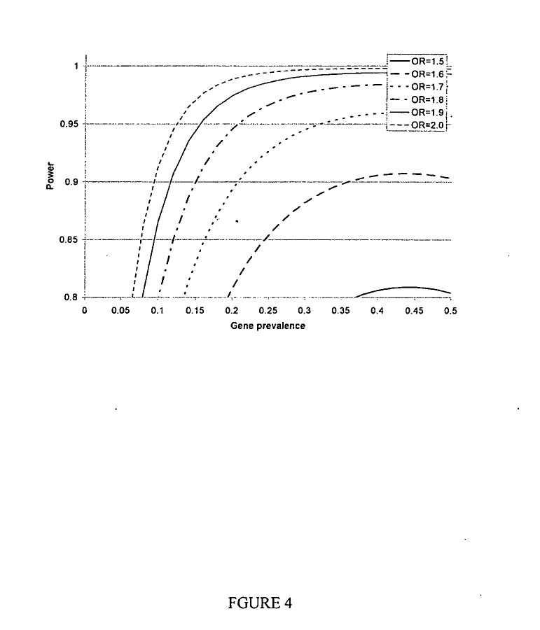

c. Power calculations. The data available for study in this project are for 288 subjects, 196 of whom do not have a diagnosis of myalgia and the remaining 92 are either definite or probable cases. The power available for detecting an odds ratio (OR) of a specified size for a particular allele was determined on the basis of a significance test on the corresponding difference in proportions using a 5% level of significance. The approach for calculating power involved the adaptation of the method given by Rosner (1995), and the results are shown in FIG. 4. The SNPs that are explored in this research are not so common as to have prevalence of more than 35%, but rather in the range of 10-15%. Therefore, it is apparent that the study has at least 80% power to detect odds ratios in the range of 1.6-1.8, which are modest effects.

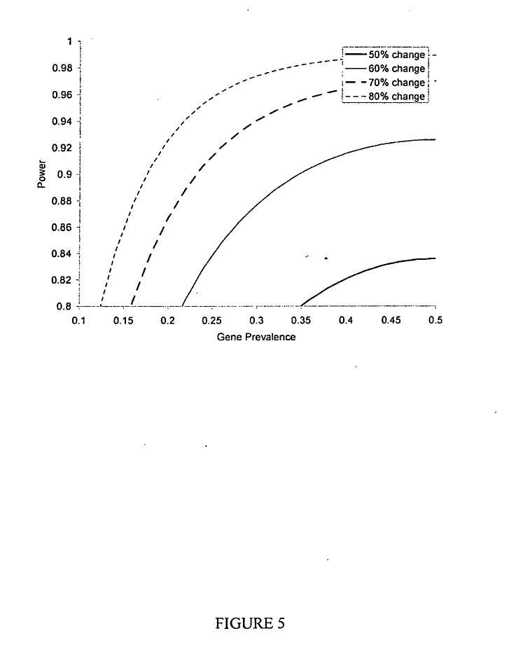

A second outcome indicative of SIM is the level of creatine kinase. These data indicates that this has a log normal distribution with standard deviation of the natural log transformed value being 0.44. Because a specified difference on the log scale corresponds to a proportional difference on the arithmetic scale, power calculations are performed that are available for detecting a proportionate change in creatine kinase. A total of 210 of these subjects are known to have valid measures of creatine kinase, and these calculations are based on the method described by Rosner (1995) using a 5% significance level for the test. Results from the calculations are shown in FIG. 5. Because the gene prevalence for the SNPs under study will be in the range of 10-20%, this study will have at least 80% power to detect a change of 60-70%.

d. Model validation. A cross-validation approach is used to evaluate the performance of models by separating the data used for parameterization (training set) from the data used for testing (test set). The approach randomly divides the population into the training set, which will comprise 80% of the subjects, and the remaining 20% will be the test set. The algorithmic approach is used for finding a model that can be used for prediction of whether myalgia or elevated CK will occur in a subject using the data in the training set. This prediction equation is then used to prepare an ROC curve that provides an independent estimate of the relationship between sensitivity and specificity for the prediction model.

### REFERENCES

1. Ahn D, Ge Y, Stricklett P K, Gill P, Taylor D, Hughes A K, Yanagisawa M, Miller L, Nelson R D, Kohan D E: Collecting duct-specific knockout of endothelin-1 causes hypertension and sodium retention. J. Clin. Invest 114: 504-511(2004).
2. Auer, J.; Berent, R.; Weber, T.; Eber, B. Clinical significance of pleiotropic effects of statins: lipid reduction and beyond. Curr Med Chem 9:1831-1850 (2002).
3. Ball S E, Scatina J, Kao J, et al. Population distribution and effects on drug metabolism of a genetic variance in the 5′ promoter region of CYP3A4. Clin Pharmacol Ther 66:288-94 (1999).
4. Beltowski, J. Adiponectin and resistin—new hormones of white adipose tissue. Med Sci Monit 9:RA55-61 (2003).
5. Benjamini Y, Hochberg Y: Controlling the false discovery rate: a practical and powerful approach to multiple testing. Journal of the Royal Statistical Society, Series B 57:289-300 (1995).
6. Benjamini Y, Hochberg Y: On the adaptive control of the false discovery rate in multiple testing with independent statistics. Journal of Educational and Behavioral Statistics 25:60-83 (2000).
7. Boden, W. E. High-density lipoprotein cholesterol as an independent risk factor in cardiovascular disease: assessing the data from Framingham to the Veterans Affairs High—Density Lipoprotein Intervention Trial. Am J Cardiol 86:19L-22L (2000).
8. Carmeliet P: Angiogenesis in health and disease. Nat. Med. 9. 653-60 (2003).
9. Clark J F: The creatine kinase system in smooth muscle. Mol Cell Biochem. April-May 133-134:221-32 (1994).
10. Cleveland, W S: Robust locally weighted regression and smoothing scatterplots. Journal of American Statistical Association 74, 829-836 (1979).
11. Cleveland W S, Devlin S J: Locally Weighted Regression: An Approach to Regression Analysis by Local Fitting. Journal of the American Statistical Association Vol. 83, pp. 596-610 (1988).
12. Crane, F. L. Biochemical functions of coenzyme Q10. J Am Coll Nutr 20:591-598 (2001).
13. Dimitroulakos J, Yeger H. HMG-CoA reductase mediates the biological effects of retinoic acid on human neuroblastoma cells: lovastatin specifically targets P-glycoprotein-expressing cells. Nat Med 2:326-33 (1996).
14. Downs J R, Clearfield M, Weis S, Whitney E, Shapiro D R, Beere P A et al.: Primary prevention of acute coronary events with lovastatin in men and women with average cholesterol levels: results of AFCAPS/TexCAPS. Air Force/Texas Coronary Atherosclerosis Prevention Study. JAMA 279(20):1615-1622 (1998).
15. Durrleman S, Simon R. Flexible regression models with cubic splines. Statistics in Medicine 8:551-561 (1989).
16. Fromm M F. The influence of MDR1 polymorism on P-glycoprotein expression and function in humans. Adv Drug Deliv Revi 2002; 54:1295-1310.
17. Fan J B, Oliphant A, Shen R, Kermani B, Garcia F, Gunderson K L, Hansen M, Steemers F, Butler B L, Deloukas P, et al. Highly parallel SNP genotyping. Cold Spr. Harb. Symp. Biol. 68 (2003).
18. Fromm M F. The influence of MDR1 polymorism on P-glycoprotein expression and function in humans. Adv Drug Deliv Revi 54:1295-1310 (2002).
19. Ghirlanda, G.; Oradei, A.; Manto, A.; Lippa, S.; Uccioli, L.; Caputo, S.; Greco, A. V.; Littarru, G. P. Evidence of plasma CoQ10-lowering effect by HMG-CoA reductase inhibitors: a double-blind, placebo-controlled study. J Clin Pharmacol 33:226-9 (1993).
20. Giacobino, J. P. Uncoupling protein 3 biological activity. Biochem Soc Trans 29:774-777 (2001).
21. Gonzalez-Cadavid, N. F.; Taylor, W. E.; Yarasheski, K.; Sinha-Hikim, I.; Ma, K.; Ezzat, S.; Shen, R.; Lalani, R.; Asa, S.; Mamita, M.; Nair, G.; Arver, S.; Bhasin, S. Organization of the human myostatin gene and expression in healthy men and HIV-infected men with muscle wasting. Proc Nat Acad Sci 95:14938-14943 (1998).
22. Guttridge, D. C.; Mayo, M. W.; Madrid, L. V.; Wang, C.-Y.; Baldwin, A. S, Jr. NF-kappa-B-induced loss of MyoD messenger RNA: possible role in muscle decay and cachexia. Science 289:2363-2366 (2000).
23. Hastie T, Tibshirani R. Generalized additive models. Stat. Sci. 1: 297-318 (1986).
24. Hoag G N, Franks C R, Smith C, DeCoteau W E: Creatine kinase isoenzyme patterns in normal smooth muscle and smooth muscle neoplasms. Clin Biochem 13(4):149-50 (1980).
25. Holash J, Wiegand S J, Yancopoulos G D: New model of tumor angiogenesis: dynamic balance between vessel regression and growth mediated by angiopoietins and VEGF. Oncogene September 20; 18(38):5356-62. Review. (1999).
26. Hsiang B, Zhu Y, Wahg Z, Wu Y, Sasseville V, et al. A novel human hepatic organic anion transporting polypeptide (OATP2). Identification of a liver-specific human organic anion transporting polypeptide and identification of rat and human hydroxymethylgluytaryl-CoA reductase inhibitor transporters. J Biol Chem 274:37161-86 (1999).
27. Imanishi T, Hano T, Sawamura T, Takarada S, Nishio I: Oxidized low density lipoprotein potentiation of Fas-induced apoptosis through lectin-like oxidized-low density lipoprotein receptor-1 in human umbilical vascular endothelial cells. Circ J. November; 66(11):1060-4 (2002).
28. Kendall R L, Wang G, Thomas K A: Identification of a natural soluble form of the vascular endothelial growth factor receptor, FLT-1, and its heterodimerization with KDR. Biochem. Biophys. Res. Commun. 226: 324-328 (1996).
29. Knapp A C, Huang J, Starling G and Kiener P A: Inhibitors of HMG-CoA reductase sensitize human smooth muscle cells to Fas-ligand and cytokine-induced cell death. Atherosclerosis 152: 217-227 (2000).
30. Kwak H J, So J N, Lee S J, Kim I, Koh G Y: Angiopoietin-1 is an apoptosis survival factor for endothelial cells. FEBS Lett. April 9; 448(2-3):249-53. (1999).
31. Laaksonen, R.; Ojala, J. P.; Tikkanen, M. J.; Himberg, J. J. Serum ubiquinone concentrations after short- and long-term treatment with HMG-CoA reductase inhibitors. Eur J Clin Pharmacol 46:313-7 (1994).
32. Linder M W, Looney S, Adams J E 3rd, Johnson N, et al. Warfarin dose adjustments based on CYP2C9 genetic polymorphisms. J Thromb Thrombolys 14:227-32 (2002).
33. McCullagh P, Nelder J A. Generalized Linear Models. London: Chapman and Hall, 1989
34. Marshal L J Ramdin, Lara S P, Brooks T, Charlton Dphil P, Shute J K: Plasminogen Activator Inhibitor-1 Supports IL-8-Mediated Neutrophil Transendothelial Migration by Inhibition of the Constitutive Shedding of Endothelial IL-8/Heparan Sulfate/Syndecan-1 Complexes. The Journal of Immunology 171: 2057-2065 (2003).
35. Murphy T J, Alexander R W, Griendling K K; Runge, M. S.; Bernstein, K. E.: Isolation of a cDNA encoding the vascular type-1 angiotensin II receptor. Nature 351: 233-236 (1991).
36. Musumeci, O.; Andreu, A. L.; Shanske, S.; Bresolin, N.; Comi, G. P.; Rothstein, R.; Schon, E. A.; DiMauro, S. Intragenic inversion of mtDNA: a new type of pathogenic mutation in a patient with mitochondrial myopathy. Am J Hum Genet 66:1900-1904 (2000)
37. Nakagami, H.; Jensen, K. S.; Liao, J. K. A novel pleiotropic effect of statins: prevention of cardiac hypertrophy by cholesterol-independent mechanisms. Ann Med 35:398-403 (2003).
38. Nielsen, J. N.; Richter, E. A. Regulation of glycogen synthase in skeletal muscle during exercise. Acta Physiol Scand 178:309-319 (2003).
39. Nishizato Y, Ieiri I, Suzuki H, et al. Polymorphisms of OATP-C (SLC21A6) and OAT3 (SLC22A8) genes: consequences for pravastatin pharmacokinetics. Clin Pharmacol Ther 73:554-65 (2003).
40. Oakes, N. D.; Furler, S. M. Evaluation of free fatty acid metabolism in vivo. Ann N Y Acad Sci 967:158-75 (2002).
41. Oliphant A, Barker D L, Stuelpnagel J R, Chee M S BeadArray technology: Enabling an accurate, cost-effective approach to high-throughput genotyping. Biotechniques 32: S56-S61 (2002).
42. Phillips P S, Haas R H, Bannykh S, Hathaway S, Gray N L, Kimura B J et al. Statin-associated myopathy with normal creatine kinase levels. Ann Intern Med 137:581-585 (2002).
43. Reinere A, Yekutiele D, Benjamini Y: Identifying differentially expressed genes using false discovery rate controlling procedures. Bioinformatics 19:368-375 (2003).
44. Ribalta, J., Vallve, J. C., Girona, J. and Masana, L. Apolipoprotein and apolipoprotein receptor genes, blood lipids and disease. Curr. Opin. Clin. Nutr. Metab. Care. 6:177-187 (2003).
45. Rosenson R S: Current overview of statin-induced myopathy. Am J Med 116(6):408-416 Review (2004).
46. Rosner B: Fundamentals of Biostatistics. Belmont, Calif.: Wadsworth Publishing Co. (1995).
47. Ruaño G, Windemuth A, Holford T: Physiogenomics: Integrating systems engineering and nanotechnology for personalized health. The Biomedical Engineering Handbook, 3rd Edition, CRC Press (2005). (in press)
48. Rubanyi G. M: Endothelium-derived relaxing and contracting factors. Journal of Cellular Biochemistry 46, 27-36 (1991).
49. Sacks F M, Pfeffer M A, Moye L A, Rouleau J L, Rutherford J D, Cole T G et al.: The effect of pravastatin on coronary events after myocardial infarction in patients with average cholesterol levels. Cholesterol and Recurrent Events Trial investigators. N Engl J Med 335, 1001-1009 (1996).
50. Saltelli A, Chan K, Scott E M: Sensitivity Analysis. John Wiley and Sons, Chichester. (2000).
51. Schmitz G, Drobnik W. Pharmacogenomics and pharmacogenetics of cholesterol-lowering therapy. Clin Chem Lab Med 41:581-9 (2003).
52. Shepherd J: Statin therapy in clinical practice: new developments. Curr Opin Lipidol 6(5):254-5 (1995).
53. Staffa J A, Chang J, Green L: Cerivastatin and reports of fatal rhabdomyolysis. N Engl J Med 346:539-540 (2002).
54. Steenland K, Bray I, Greenland S, Boffetta P. Empirical Bayes adjustments for multiple results in hypothesis-generating or surveillance studies. Ca Epidemiol Biomarkers Prev. 9:895-903 (2000).
55. Thompson, P D, Clarkson, P, and Karas, R H: Statin-associated myopathy. JAMA 289, 1681-90 (2003).
56. Tirona R G, Kim R B. Pharmacogenomics of organic anion-transporting polypeptides (OATP). Adv Drug Del Reviews 54:1343-52 (2002).
57. von Ahsen N, Richter M, Grupp C, Ringe B, Oellerick M, Armstron V W. No influence of the MDR-1 C3435T polymorphism or a CYP3A4 promoter polymorphism (CYP3A4-V allele) on dose-adjusted cyclosporin A trough concentrations or rejection incidence in stable renal transplant recipients. Clin Chem 47:1048-52 (2001).
58. Wang, T. D.; Chen, W. J.; Lin, J. W.; Cheng, C. C.; Chen, M. F.; Lee, Y. T. Efficacy of fenofibrate and simvastatin on endothelial function and inflammatory markers in patients with combined hyperlipidemia: relations with baseline lipid profiles. Atherosclerosis 170:315-323 (2003).
59. Wilke R A, Moore J H, Burmester J K: Relative impact of CYP3A genotype and concomitant medication on the severity of atorvastatin-induced muscle damage. Pharmacogenet Genomics 15(6):415-21 (2005).
60. Wolf C R, Smith G. Pharmacogenetics. Br Med Bull 55:366-86 (1999).
61. Yamazaki M, Akiyama S, Ni'inuma K, et al. Biliary excretion of pravastatin in rats: contribution of the excretion pathway mediated by canalicular multispecific organ anion transporter. Drug Metab Disp 25:1123-9 (1997).
62. Yates C R, Zhang W, Song P, Li S, et al. The effect of CYP3A5 and MDR1 polymorphic expression on cyclosporine oral disposition in renal transplant patients. J Clin Pharmacol 43:555-64 (2003).
63. Zöliner S, Haseloff R F, Kirilyuk I A, Blasig I E, Rubanyi G M: Nitroxides increase the detectable amount of nitric oxide released from endothelial cells. J. Biol. Chem 272 23076-80 (1997).

We claim:

1. An ensemble of marker genes useful for determining an individual at risk for muscle injury and/or muscular side effects in response to statin treatment comprising at least two single nucleotide polymorph (SNP) gene variants selected from the group consisting of: rs2933249; rs12695902; rs1549758; rs1799983; rs1800808; rs6136; rs6131; rs6092; rs5361; rs2742115; rs5369; rs877172; rs1283718; rs2514869; rs1283694; rs1570679; rs2296189; rs10507383; rs748253; rs675; rs2740574; rs1800716; rs2020933; rs2070424; rs854572; rs3756450; rs600728; rs3176921; rs10841044; rs7200210; rs5491; rs617333; rs2058112; rs1800794; rs504714; rs6195; rs1042718; rs2276307; rs7412; rs6488950; rs9904270; rs2049045; rs6265; rs132653; rs6318; and rs2838549.

2. An array comprising the ensemble of gene markers of claim 1.

3. A solid support comprising the array of claim 2.

4. A method for determining an individual at risk for muscle injury and/or muscular side effects in response to statin treatment comprising (1) obtaining genetic material from said individual; and (2) assaying said genetic material for the presence of said at least two SNP gene variants of the ensemble of claim 1.

5. The method of claim 4 wherein the at least two SNP gene variants comprise at least one SNP with a positive coefficient and at least one SNP with a negative coefficient.

6. An ensemble of marker genes useful for determining an individual at risk for muscle injury and/or muscular side effects in response to statin treatment comprising:
at least two SNP gene variants, the presence of which correlates with at least one statin injury to muscle and muscle side effects in humans;
wherein said injury is selected from the group consisting of log concentration of serum creatine kinase and myalgia; and combinations thereof; and
(a) in the case where said injury is the log concentration of serum creatine kinase, said ensemble of marker genes comprises rs1799983; rs877172; rs675; rs12695902; rs2740574; rs1800716; rs2020933; rs2296189; rs2070424; rs854572; rs3756450; rs1611115; rs600728; rs3176921; rs10841044; rs7200210; rs5491; rs617333; rs1549758; and rs2514869; and
(b) in the case where said injury is myalgia, said ensemble of marker genes comprises rs2058112; rs1800794; rs504714; rs6195; rs2742115; rs1042718; rs2276307; rs7412; rs6488950; rs9904270; rs1570679; rs2049045; rs6265; rs132653; rs6318; and rs2838549.

7. An array comprising the ensemble of gene markers of claim 6.

8. A solid support comprising the array of claim 7.

9. A method for determining an individual at risk for muscle injury and/or muscular side effects in response to statin treatment comprising (1) obtaining genetic material from said individual; and (2) assaying said genetic material for the presence of said at least two SNP gene variants of the ensemble of claim 5.

10. The method of claim 9 wherein the at least two SNP gene variants comprise at least one SNP with a positive coefficient and at least one SNP with a negative coefficient.

11. A marker gene set comprising at least one genetic variant of a marker gene associated with muscle injury and/or muscular side effects in response to statin treatment, wherein the genetic variant is within the marker genes selected from the group consisting of NOS3, OXT, APOA4, AGTR1, CYP3A4, CYP2D6, SLC6A4, FLT1, SOD1, OPN1, SLC6A3, DBH, TEK, CRH, PIK3C2G, SLC12A4, ICAM1, ADIPOR2, IL1A, AVEN, NR3C1, OLR1, ADRB2, HTR3B, APOE, SCARB1, RARA, FLT1, BDNF, APO3L, HTR2C, EDN, SELP, SELE, SERPINE1, and PFKL.
